(19) United States
(12) Patent Application Publication
(10) Pub. No.: US 2024/0402147 A1
(43) Pub. Date: Dec. 5, 2024

(54) METHOD OF ESTABLISHING CANCER SCREENING MODULE, USING METHOD AND PLATFORM THEREOF

(71) Applicant: NATIONAL TAIWAN UNIVERSITY, TAIPEI (TW)

(72) Inventors: Cheng-Chih HSU, TAIPEI (TW); Hou-Chun HUANG, TAIPEI (TW); Hsin-Hsiang CHUNG, TAIPEI (TW); Laura Min Xuan CHAI, TAIPEI (TW); Yi-Hsin CHEN, TAIPEI (TW); Jia-Ying YU, TAIPEI (TW); Ming-Yang WANG, TAIPEI (TW)

(21) Appl. No.: 18/675,373

(22) Filed: May 28, 2024

Related U.S. Application Data
(60) Provisional application No. 63/469,517, filed on May 29, 2023.

(30) Foreign Application Priority Data
Jan. 22, 2024 (TW) 113102480

(51) Int. Cl. G01N 33/483; A61B 10/02; H01J 49/04; H01J 49/42

(52) U.S. Cl. CPC G01N 33/4833; H01J 49/0445; H01J 49/42; A61B 10/0233

(57) ABSTRACT

A method of establishing a cancer screening model is provided, including: providing a plurality of samples and a plurality of corresponding cancer states; analyzing these samples by a low-resolution mass spectrometer to obtain a plurality of mass spectral data, wherein the low-resolution mass spectrometer is undertaken a mass accuracy level above 5 ppm and a mass resolution (m/Δm) below 10,000; inputting these mass spectral data into a machine learning algorithm to obtain a plurality of markers by a feature selection method; and using these markers and these cancer states by the machine learning algorithms to establish cancer screening model.

### CROSS-REFERENCE TO RELATED APPLICATION

This application claims priority to U.S. Provisional Application Ser. No. 63/469,517 filed on May 29, 2023, and Taiwan Application Serial Number 113102480, filed on Jan. 22, 2024, the disclosures of which are incorporated herein by reference in their entireties.

### BACKGROUND

### Field of Invention

The present invention relates to a method and a platform thereof. More particularly, the present invention relates to a method of establishing cancer screening module by a low-resolution mass spectrometer, a using method and a platform thereof.

### Description of Related Art

Core needle biopsy (CNB) has been widely conducted as the standard procedure to acquire biopsy for breast cancer diagnosis in hospitals. However, the conventional histological examination of CNB is time- and labor-intensive. The examination process includes formalin-fixed paraffin-embedded tissue preparation, tissue sectioning, and multiple staining procedures such as hematoxylin and eosin and immunohistochemistry (IHC). The stained tissue sections are then evaluated by pathologists under a microscope.

Though the CNB examination is routinely performed in hospital, it normally requires several days for a conclusive report. The delayed diagnostic reports usually increase the psychological burden of patients and may result in poorer prognosis. Furthermore, because of the heterogeneity of histological features, diagnostic discordance could occur between different cohorts and pathologists, influencing subsequent clinical treatments.

Therefore, how to establish a rapid and objective platform for cancer diagnosis, and the related art really needs to be improved.

### SUMMARY

The purpose of the present disclosure is to use a low-resolution mass spectrometer combined with a machine learning algorithm for designing a simple-to-operate screening platform thereby quickly screening cancer. Cancer screening using untargeted metabolomics through the low-resolution mass spectrometry is an unprecedented approach that can assist with intraoperative and treatment decision-making.

The invention provides a method of establishing cancer screening module, comprising: providing a plurality of samples and a plurality of cancer statuses corresponding to the plurality of samples; analyzing the plurality of samples with a low-resolution mass spectrometer to obtain a plurality of mass spectral data, wherein the low-resolution mass spectrometer is a mass spectrometer with mass accuracy level above 5 ppm and a mass resolution below 10,000 m/Δm; inputting the plurality of mass spectral data into a machine learning algorithm to obtain a plurality of markers by a feature selection method; and using the plurality of markers and the plurality of cancer statuses to establish the cancer screening model by the machine learning algorithm.

In some embodiments, the inputting of the plurality of mass spectral data into the machine learning algorithm comprises: obtaining a plurality of initial markers from the plurality of mass spectral data by an initial feature selection method, and then inputting the plurality of initial markers into the machine learning algorithm, wherein the initial feature selection method comprises filter method.

In some embodiments, when the feature selection method is wrapper method or embedded method, the feature selection method comprises splitting the plurality of samples into a training set and a validation set, calculating a sensitivity, a specificity, an accuracy, an area under curve (AUC) of a receiver operating characteristic (ROC), or a combination thereof to obtain the plurality of markers.

In some embodiments, the wrapper method comprises a recursive feature elimination (RFE) to obtain the plurality of markers.

In some embodiments, before the inputting of the plurality of mass spectral data into the machine learning algorithm, the method further comprises performing a normalized preprocessing on the plurality of mass spectral data, the normalization preprocessing comprises: normalization, m/z alignment, average MS spectra, m/z binning, noise removal, data scaling, or a combination thereof.

In some embodiments, the m/z binning comprises binning by size from 0.5 daltons to 1.5 daltons.

In some embodiments, the low-resolution mass spectrometer is single quadrupole mass spectrometer, wherein the mass accuracy level is from 5 ppm to 1200 ppm and the mass resolution is below 10,000 m/m.

In some embodiments, the machine learning algorithm comprises kernel-based, regression, tree-based, dimension reduction, probabilistic, distance-based, or any combination thereof.

In some embodiments, the kernel-based comprises support vector machine (SVM), when the plurality of samples is a plurality of benign breast tumor samples and a plurality of malignant breast tumor samples, the SVM is used to analyze the plurality of cancer statuses being benign breast tumor or malignant breast tumor; when the plurality of samples are a plurality of HR (hormone receptor) negative breast cancer tumor samples and a plurality of HR positive breast cancer tumor samples, the SVM is used to analyze the plurality of cancer statuses being HR negative breast cancer tumor or HR positive breast cancer tumor; when the plurality of samples are a plurality of HER2 (human epidermal growth factor receptor 2) negative breast cancer tumor samples and a plurality of HER2 positive breast cancer tumor samples, the SVM is used to analyze the plurality of cancer statuses being HER2 negative breast cancer tumor or HER2 positive breast cancer tumor; when the plurality of samples comprise a plurality of HR negative breast cancer tumor samples, a plurality of HER2 negative breast cancer tumor samples, a plurality of HR positive breast cancer tumor samples and a plurality of HER2 positive breast cancer tumor samples, the SVM is used to analyze the plurality of cancer statuses being HR negative HER2 negative breast cancer tumor, HR negative HER2 positive breast cancer tumor, HR positive HER2 negative breast cancer tumor, or HR positive HER2 positive breast cancer tumor; when the plurality of samples are a plurality of normal lymph node samples and a plurality of samples of lymph node with metastatic breast cancer cells, the SVM is used to analyze the plurality of cancer statuses being lymph node without breast cancer metastasis or lymph node with breast cancer metastasis; when the plurality of samples are a plurality of samples of normal surgical margins of the breast and a plurality of breast cancer tissue samples, the SVM is used to analyze the plurality of cancer statuses being normal breast tissue or breast cancer tissue; when the plurality of samples are a plurality of normal skin tissue samples and squamous cell carcinoma samples, the SVM is used to analyze the plurality of cancer statuses being normal skin tissue or squamous cell carcinoma; or when the plurality of samples are a plurality of follicular thyroid carcinoma samples and a plurality of papillary thyroid carcinoma samples, the SVM is used to analyze the plurality of cancer statuses being follicular thyroid carcinoma or papillary thyroid carcinoma.

In some embodiments, wherein when the plurality of samples are the plurality of benign breast tumor samples and the plurality of malignant breast tumor samples, the SVM comprises SVM-RFE to obtain the plurality of markers, wherein the plurality of markers are selected from any one or any combination of the group consisting of m/z 782.5, m/z 798.5, m/z 754.5, m/z 770.5, m/z 923.5, m/z 772.5, m/z 757.5, m/z 774.5, m/z 788.5, m/z 753.5, and m/z 727.5.

In some embodiments, wherein when the plurality of samples are the plurality of samples of normal surgical margins of the breast and the plurality of breast cancer tumor samples, the SVM comprises SVM-RFE to obtain the plurality of markers, wherein the plurality of markers are selected from any one or any combination of the group consisting of m/z 504.5, m/z 835.5, m/z 575.5, m/z 723.5, m/z 764.5, m/z 837.5, m/z 804.5, m/z 547.5, m/z 853.5, m/z 765.5, m/z 788.5, m/z 828.5, m/z 781.5, m/z 727.5, m/z 759.5, m/z 836.5, m/z 682.5, m/z 753.5, m/z 786.5, m/z 770.5, m/z 805.5, m/z 518.5, m/z 768.5, m/z 755.5, m/z 782.5, m/z 756.5, m/z 824.5, m/z 754.5, m/z 647.5, and m/z 848.5.

In some embodiments, wherein when the plurality of samples are the plurality of normal lymph node samples and the plurality of samples of lymph node with metastatic breast cancer cells, the SVM comprises SVM-RFE to obtain the plurality of markers, wherein the plurality of markers are selected from any one or any combination of the group consisting of m/z 509.5, m/z 531.5, m/z 534.5, m/z 567.5, m/z 575.5, m/z 615.5, m/z 641.5, m/z 643.5, m/z 698.5, m/z 742.5, m/z 754.5, m/z 758.5, m/z 761.5, m/z 781.5, m/z 782.5, m/z 797.5, m/z 798.5, m/z 805.5, m/z 820.5, m/z 824.5, m/z 828.5, m/z 829.5, m/z 830.5, m/z 846.5, m/z 879.5, and m/z 880.5.

In some embodiments, wherein when the plurality of samples are the plurality of normal skin tissue samples and the plurality of squamous cell carcinoma samples, the SVM comprises SVM-RFE to obtain the plurality of markers, wherein the plurality of markers are selected from any one or any combination of the group consisting of m/z 734.5, m/z 782.5, m/z 735.5, m/z 796.5, m/z 798.5, m/z 756.5, m/z 780.5, m/z 758.5, m/z 786.5, m/z 766.5, m/z 813.5, and m/z 814.5.

In some embodiments, the regression comprises LASSO regression, when the plurality of samples are a plurality of normal skin tissue samples, a plurality of benign nevus tissue samples and a plurality of melanoma samples, the LASSO regression is used to analyze the plurality of cancer statuses being normal skin tissue or melanoma; when the plurality of samples are a plurality of normal lymph node samples and a plurality of samples of lymph node with metastatic melanoma cells, the LASSO regression is used to analyze the plurality of cancer statuses being lymph node without metastatic melanoma or lymph node with metastatic melanoma; when the plurality of samples are a plurality of normal skin tissue samples and a plurality of basal cell carcinoma samples, the LASSO regression is used to analyze the plurality of cancer statuses being normal skin tissue or basal cell carcinoma; or when the plurality of samples are a plurality of benign thyroid nodule samples and a plurality of malignant thyroid nodule samples, the LASSO regression is used to analyze the plurality of cancer statuses being benign thyroid nodule or malignant thyroid nodule.

In some embodiments, when the plurality of samples are the plurality of normal skin tissue samples, the plurality of benign nevus tissue samples and the plurality of melanoma samples, the LASSO regression comprises LASSO regression feature selection to obtain the plurality of markers, wherein the plurality of markers are selected from any one or any combination of the group consisting of m/z 766.5, m/z 664.5, m/z 728.5, m/z 729.5, m/z 773.5, m/z 719.5, m/z 692.5, m/z 752.5, m/z 757.5, m/z 736.5, m/z 862.5, m/z 672.5, m/z 603.5, m/z 832.5, and m/z 521.5.

In some embodiments, when the plurality of samples are the plurality of normal lymph node samples and the plurality of samples of lymph node with metastatic melanoma cells, the LASSO regression comprises LASSO regression feature selection to obtain the plurality of markers, wherein the plurality of markers are selected from any one or any combination of the group consisting of m/z 700.5, m/z 761.5, m/z 771.5, m/z 732.5, m/z 708.5, m/z 817.5, and m/z 622.5.

In some embodiments, when the plurality of samples are the plurality of normal skin tissue samples and the plurality of basal cell carcinoma samples, the LASSO regression comprises LASSO regression feature selection to obtain the plurality of markers, wherein the plurality of markers are selected from any one or any combination of the group consisting of m/z 846.5, m/z 719.5, m/z 751.5, m/z 774.5, m/z 780.5, m/z 814.5, m/z 813.5, m/z 759.5, m/z 766.5, m/z 692.5, m/z 888.5, m/z 731.5, m/z 677.5, m/z 704.5, m/z 772.5, m/z 804.5, m/z 744.5, m/z 781.5, m/z 702.5, m/z 716.5, m/z 830.5, m/z 908.5, m/z 783.5, m/z 696.5, m/z 890.5, m/z 896.5, m/z 784.5, m/z 912.5, and m/z 826.5.

In some embodiments, the inputting the plurality of mass spectral data into the machine learning algorithm comprises: obtaining a plurality of initial markers from the plurality of mass spectral data by an initial feature selection method, and then inputting the plurality of initial markers into the machine learning algorithm, wherein the initial feature selection method comprises filter method, wherein when the plurality of samples are the plurality of normal skin tissue samples and the plurality of basal cell carcinoma samples, the LASSO regression comprises LASSO regression feature selection to obtain the plurality of markers, wherein the plurality of markers are selected from any one or any combination of the group consisting of m/z 706.5, m/z 799.5, m/z 826.5, m/z 770.5, m/z 825.5, m/z 798.5, m/z 787.5, m/z 771.5, m/z 707.5, m/z 768.5, m/z 863.5, m/z 786.5, m/z 838.5, m/z 703.5, m/z 772.5, m/z 744.5, m/z 730.5, m/z 816.5, m/z 721.5, m/z 823.5, m/z 736.5, m/z 820.5, m/z 766.5, m/z 861.5, m/z 702.5, m/z 814.5, m/z 833.5, m/z 689.5, m/z 759.5, m/z 756.5, m/z 778.5, m/z 745.5, m/z 830.5, and m/z 750.5.

In some embodiments, when the plurality of samples are the plurality of benign thyroid nodule samples and the plurality of malignant thyroid nodule samples, the LASSO regression comprises LASSO regression feature selection to obtain the plurality of markers, wherein the plurality of markers are selected from any one or any combination of the group consisting of m/z 741.5, m/z 799.5, m/z 782.5, m/z 770.5, m/z 961.5, m/z 929.5, m/z 572.5, m/z 771.5, m/z 871.5, m/z 696.5, m/z 743.5, m/z 843.5, m/z 761.5, m/z 667.5, m/z 980.5, m/z 508.5, m/z 916.5, m/z 764.5, m/z 904.5, m/z 734.5, m/z 557.5, m/z 673.5, m/z 922.5, m/z 561.5, m/z 957.5, m/z 573.5, m/z 813.5, m/z 903.5, m/z 507.5, m/z 896.5, m/z 989.5, m/z 769.5, m/z 803.5, m/z 566.5, m/z 660.5, m/z 528.5, m/z 802.5, m/z 621.5, m/z 809.5, m/z 534.5, m/z 854.5, m/z 926.5, m/z 738.5, m/z 719.5, m/z 969.5, m/z 825.5, m/z 754.5, m/z 747.5, m/z 590.5, and m/z 781.5.

In some embodiments, the analyzing the plurality of samples with the low-resolution mass spectrometer comprises: ionizing the plurality of samples by a paper spray ionization (PSI) method; and analyzing the plurality of samples from the plurality of ionized samples by the low-resolution mass spectrometer.

In some embodiments, the PSI method comprises: using a PSI device comprising: a base; a solvent rack disposed above the base; and a clamp having a fixing end and a clamping end, the fixing end disposed on the base; placing one of a plurality of paper sheets to a clamping end of the clamp; placing a solvent to the solvent rack of the PSI device; placing the plurality of samples on the different plurality of paper sheets, and using the solvent to perform PSI to obtain a plurality of ionized substance; and collecting the plurality of ionized substance and analyzing the plurality of samples with the low-resolution mass spectrometer.

In some embodiments, the PSI method comprises: using a PSI device, the PSI device comprising: a base; an abutting member disposed on the base; a loading plate movably disposed on the base, the loading plate comprising: a body having a bottom surface and a side surface adjacent to the bottom surface, the side surface movably abutting the abutting member, and the bottom surface movably abutting the base; a protrusion protruding outward from the bottom surface of the body; and a metal placing piece disposed on the body and the protrusion; a solvent rack disposed on the base; and placing one of a plurality of paper sheets on the metal placing piece, and a corner of the one of the plurality of paper sheets protruding outward from the protrusion, and a protruding direction of the corner and a protruding direction of the protrusion being the same and facing toward the base; placing a solvent to the solvent rack of the PSI device; placing the plurality of samples on the different plurality of paper sheets respectively, and using the solvent to perform PSI to obtain a plurality of ionized substance; and collecting the plurality of ionized substance and analyzing the plurality of samples with the low-resolution mass spectrometer.

The present disclosure provides a method for cancer screening using the cancer screening model as above mentioned, comprising: providing a specimen of a subject; analyzing the specimen by the low-resolution mass spectrometer to obtain a subject mass spectral data; and inputting the subject mass spectral data into the cancer screening model to perform calculation, comparison, and evaluating a risk of a cancer for the subject. In some embodiments, the subject is human.

In some embodiments, the cancer comprises breast cancer, thyroid cancer, or skin cancer.

The present disclosure provides a cancer screening platform, comprising: a low-resolution mass spectrometer configured to analyze a plurality of samples to obtain a plurality of mass spectral data, wherein the low-resolution mass spectrometer is a mass spectrometer with mass accuracy level above 5 ppm and a mass resolution below 10,000 m/m; a cancer screening model comprising a computer processor and a memory, the memory storing a plurality of computer program instructions that, when executed by the computer processor, cause the computer processor to implement following steps, comprising: inputting the plurality of mass spectral data into a machine learning algorithm to obtain a plurality of markers by a feature selection method; and using the plurality of markers and a plurality of cancer statuses corresponding to the plurality of samples to establish the cancer screening model by the machine learning algorithm.

In some embodiments, the cancer screening platform further comprises the PSI device as above mentioned.

In some embodiments, the low-resolution mass spectrometer is configured to analyze a specimen to obtain a subject mass spectral data; the cancer screening model is executed by the computer processor, cause the computer processor to implement following steps, further comprising: inputting the subject mass spectral data into the cancer screening model to perform calculation, comparison, and evaluating a risk of a cancer for the subject.

The present disclosure provides a cancer screening platform, comprising: a low-resolution mass spectrometer configured to analyze a specimen of a subject to obtain a subject mass spectral data, wherein the low-resolution mass spectrometer is a mass spectrometer with mass accuracy level above 5 ppm and a mass resolution below 10,000 m/m; and a cancer screening model comprising a computer processor and a memory, the memory storing a plurality of computer program instructions that, when executed by the computer processor, cause the computer processor to implement following steps, comprising: inputting at least one known marker; inputting the subject mass spectral data; and comparing the at least one known marker and the subject mass spectral data to evaluating a risk of a cancer for the subject.

In some embodiments, the at least one known marker is obtain from a high-resolution mass spectrometer being able to provide mass accuracy level below or equal to 5 ppm and a mass resolution above or equal to 10,000 (m/Δm, full width at half-maximum height, FWHM), or a low-resolution mass spectrometer being able to provide mass accuracy level above 5 ppm and a mass resolution below to 10,000 (m/Δm, FWHM).

In some embodiments, the cancer screening model is executed by the computer processor, cause the computer processor to implement following steps, further comprising: performing a normalized preprocessing on the subject mass spectral data, the normalization preprocessing comprising: normalization, m/z alignment, average MS spectra, m/z binning, noise removal, data scaling, or a combination thereof.

In some embodiments, the m/z binning comprises binning by size from 0.5 daltons to 1.5 daltons.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 is a flow chart of a method of establishing cancer screening module according to some embodiments of the present disclosure.

FIG. 2A is a schematic view of a simply constructed PSI device according to some embodiments of the present disclosure.

FIGS. 2B to 2F are schematic views of a PSI device according to some embodiments of the present disclosure.

FIGS. 2G to 2I are schematic views of a PSI device according to another embodiment of the present disclosure.

FIGS. 3A to 3B are mass spectrum of benign and malignant breast tumor biopsy according to some embodiments of the present disclosure.

FIG. 3C is the prediction performances of benign and malignant breast tumor biopsy analyzed by different machine learning algorithm according to some embodiments of the present disclosure.

FIG. 3D is the model performance of benign and malignant breast tumor biopsy trained and model optimized through 5-fold cross validation using SVM according to some embodiments of the present disclosure.

FIG. 3E is the model performance of benign and malignant breast tumor biopsy trained through 5-fold cross validation and optimized through support vector machine recursive feature elimination (SVM-RFE) according to some embodiments of the present disclosure.

FIGS. 3F to 3G are a weighted feature table of benign and malignant breast tumor biopsy according to some embodiments of the present disclosure. “a” refers to the putative ID of compounds, assigned by referring to its exact mass (mass error within 5 ppm), and fragmentation pattern of the tandem mass spectrum; “b” refers to putative ID of compounds, assigned by referring to its exact mass (mass error within 5 ppm).

FIG. 3H is an average area under curve (AUC) of receiver operating characteristic (ROC) curve of the SVM-RFE model for distinguishing benign and malignant breast tumor biopsy according to some embodiments of the present disclosure. SD refers to standard deviation.

FIGS. 3I˜3K are the performance of the screening model updated every six months according to some embodiments of the present disclosure.

FIGS. 4A˜4C are prediction results of breast cancer molecular subtypes according to some embodiments of the present disclosure.

FIGS. 4D to 4E are weighted feature tables of normal lymph node tissue and breast cancer cells metastasis in lymph node tissue according to some embodiments of the present disclosure. “a” refers to putative ID of compounds, assigned by referring to its exact mass (mass error within 5 ppm), and fragmentation pattern of the tandem mass spectrum; “b” refers to putative ID of compounds, assigned by referring to its exact mass (mass error within 5 ppm). FIGS. 5A to 12 below all have the same symbolic meanings, so they will not be described again.

FIGS. 5A to 5C are weighted feature tables of normal breast tissue and malignant tumor at breast surgical margins according to some embodiments of the present disclosure.

FIG. 6 is a weighted feature table of normal skin tissue and melanoma according to some embodiments of the present disclosure.

FIG. 7 is a weighted feature table of normal lymph node and melanoma cancer cell metastasis in lymph nodes according to some embodiments of the present disclosure.

FIG. 8 is a feature table of normal skin tissue and basal cell carcinoma by initial feature selection method according to some embodiments of the present disclosure. “*” refers to p value less than 0.05; “**” refers to p value less than 0.01; “***” refers to p value less than 0.005; “****” refers to p value less than 0.001.

FIG. 9 is a weighted feature table of normal skin tissue and basal cell carcinoma according to some embodiments of the present disclosure.

FIG. 10 is a weighted feature table of normal skin tissue and squamous cell carcinoma according to some embodiments of the present disclosure.

FIGS. 11A to 11B are weighted feature tables of benign and malignant thyroid nodule biopsy according to some embodiments of the present disclosure.

FIGS. 12A to 12C are weighted feature tables of follicular thyroid carcinoma and papillary thyroid carcinoma tissue biopsy according to some embodiments of the present disclosure.

FIG. 13 is weighted feature tables of features selected by the machine learning model constructed with high-resolution mass spectrometry data according to some embodiments of the present disclosure.

FIG. 14A is the boxplot of m/z 798.5 abundance in malignant and benign breast tumor biopsies according to some embodiments of the present disclosure.

FIG. 14B is the ROC curve with average AUC using m/z 798.5 of benign and malignant breast tumor biopsies as a classification feature according to some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

A number of examples are provided herein to elaborate the method of establishing cancer screening module, using method and platform thereof of the instant disclosure. However, the examples are for demonstration purpose alone, and the instant disclosure is not limited thereto.

Although a series of operations or steps are used below to describe the method disclosed herein, an order of these operations or steps should not be construed as a limitation to the present disclosure. For example, some operations or steps may be performed in a different order and/or other steps may be performed at the same time. In addition, all shown operations, steps and/or features are not required to be executed to implement an embodiment of the present disclosure. In addition, each operation or step described herein may include a plurality of sub-steps or actions.

The purpose of the present disclosure is to use a low-resolution mass spectrometer combined with a machine learning algorithm for designing a simple-to-operate screening platform thereby quickly screening cancer. Cancer screening using untargeted metabolomics through the low-resolution mass spectrometry is an unprecedented approach that can make intraoperative and treatment decisions faster.

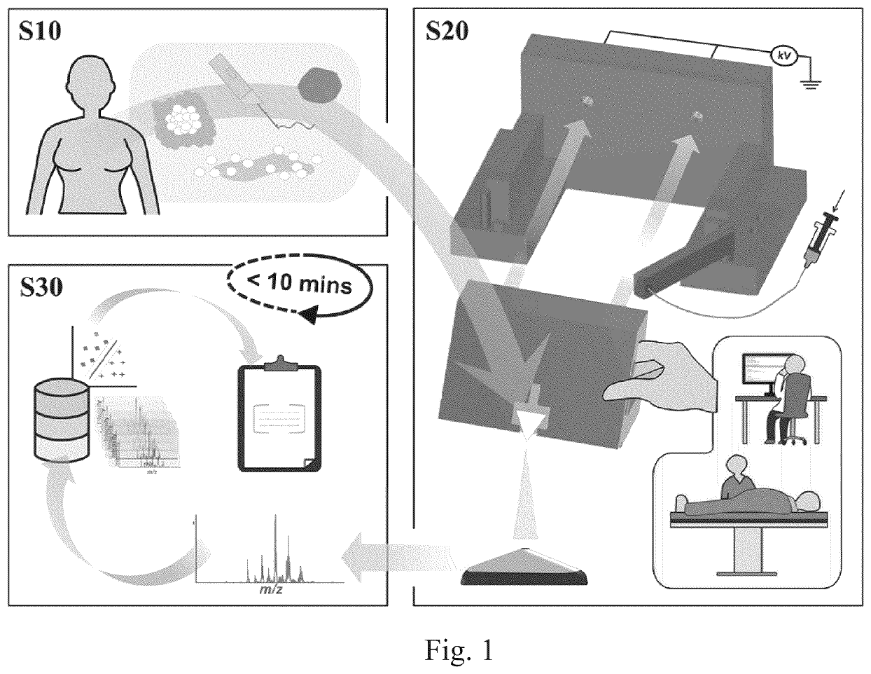

Please refer to FIG. 1, FIG. 1 is a flow chart of a method of establishing cancer screening module according to some embodiments of the present disclosure. Step S10, providing a plurality of samples and a plurality of cancer statuses corresponding to the plurality of samples. Step S20, analyzing the plurality of samples with a low-resolution mass spectrometer to obtain a plurality of mass spectral data. Step S30, inputting the plurality of mass spectral data into a machine learning algorithm to obtain a plurality of markers by a feature selection method. Using the plurality of markers and the plurality of cancer statuses to establish the cancer screening model by the machine learning algorithm.

In some embodiments, the present disclosure provides a method of establishing cancer screening module, comprises providing the plurality of samples and the plurality of cancer statuses corresponding to the plurality of samples. In some examples, the samples includes, but is not limited to normal tissue sample, benign control sample (such as nevus), benign carcinoma sample, malignant carcinoma sample, cancer without metastasis in lymph node tissue sample, cancer with metastasis in lymph node tissue sample, carcinoma margin normal sample, or a combination thereof. As shown in step S10.

In some embodiments, the present disclosure provides a method of establishing cancer screening module, comprises analyzing the plurality of samples with the low-resolution mass spectrometer to obtain the plurality of mass spectral data. As shown in step S20.

As used herein, low-resolution mass spectrometer (LRMS) refers to a mass spectrometer of mass accuracy level above 5 ppm and a mass resolution below 10,000 m/Δm (full width at half-maximum height, FWHM). Mass accuracy is determined through ppm error: (measured mass-theoretical mass)/theoretical mass. The mass resolution is the capacity of a mass spectrometer to separate ions of close m/z ratios. It is defined as the ratio of the measured mass “m” to “Δm”, the full width of the peak at half its maximum height (i.e., m/Δm, FWHM). Mass analysers of LRMS can typically be categorized as ion-trap (IT)-based or quadrupole (Q)-based mass analysers. A combination of both analysers (e.g. Q-IT) or several of the same mass analyser (e.g. QqQ) would also be possible. There are currently various forms of IT-based mass analysers. It could be a linear, rectilinear or cylindrical ion trap. On the other hand, quadrupole-based mass analysers are usually single or triple quad (QqQ). Else, they would be coupled with ITs such as quadrupole ion traps. Furthermore, as the field is continuously developing, these analysers would one day able to achieve more than what it is capable of now.

In some examples, low-resolution mass spectrometer is single quadrupole mass spectrometer, in which the mass accuracy level is from 5 ppm to 1200 ppm and the mass resolution is lower than 10,000 m/Δm.

As used herein, high-resolution mass spectrometers (HRMS) can be clearly defined as: mass spectrometers that are able to provide mass accuracy level below or equal to 5 ppm, and a mass resolution above or equal to 10,000 (m/Δm, FWHM). The type of mass analyser usually determines if the mass spectrometer is capable of acquiring high-resolution data. For instance, OrbiTrap, Fourier-transform ion cyclotron resonance (FT-ICR), and time-of-flight (TOF) instruments, in their optimum condition, will produce data that fulfils the criteria of HRMS.

### Machine Learning Model Construction

### 1. Data Pre-Processing

In some embodiments, before inputting the mass spectral data into the machine learning algorithm, the method further comprises performing a normalized preprocessing on the plurality of mass spectral data, the normalization preprocessing comprises: normalization, m/z alignment, average MS spectra, m/z binning, noise removal, data scaling, or a combination thereof.

In some examples, raw data had to be first converted into formats readable by intended processing programs (e.g. .csv, .cdf, etc.). And methods commonly utilized for processing MS spectra can then be employed, which includes, but is not limited to normalization (e.g. total ion chromatogram (TIC), base peak, endogenous compounds (as the correction of Y-axis), m/z alignment (as the correction of X-axis), average MS spectra (to increase spectrum signal-to-noise ratio), m/z binning (as the resolution of MS, bin size is from 0.5 daltons (Da) to 1.5 Da, such as 0.5 Da, 0.6 Da, 0.7 Da, 0.8 Da, 0.9 Da, 1.0 Da, 1.1 Da, 1.2 Da, 1.3 Da, 1.4 Da, 1.5 Da, or any value between any two of these values), noise removal, data scaling, or a combination thereof. Even if the structure of the endogenous compound is unknown, it can be used as a basis for normalization.

In some examples, during the normalization method, TIC (Total Ion Current) normalization involves respectively dividing all ions in the mass spectrum by the sum of all ion intensities within the mass spectra; the base peak normalization refers to normalization using the most intense peak in the mass spectrum, where each peak intensity is divided by the intensity of the base peak to; endogenous compounds, which are endogenously present in samples, usually in fixed amounts, can be used as a basis for normalization.

### 2. Data Splitting

In some examples, the samples were split into a training set and a validation set. Training and test sets were randomly conducted in a set ratio, including but not limited to 1:9˜9:1, such as 1:9, 2:8, 3:7, 4:6, 5:5, 6:4, 7:3, 8:2, 9:1, or any ratio between any two of these ratios. Another split for an external validation set may also be included, but not limited to 7:2:1.

### 3. Model Selection

In some embodiments, the present disclosure provides a method of establishing cancer screening module, comprising inputting the plurality of mass spectral data into the machine learning algorithm by a feature selection method to obtain a plurality of markers.

In some embodiments, the machine learning algorithm includes, but is not limited to kernel-based, regression, tree-based, dimension reduction, probabilistic, distance-based or any combination thereof. In one embodiment, the kernel-based algorithms includes SVM; the regression algorithms includes regression analysis, such as LASSO regression etc.

### 4. Model Optimization

In some examples, the feature selection method includes splitting the samples into the training set and the validation set, and utilizing SVM-RFE to obtain the markers. In some examples, the RFE includes calculating a sensitivity, a specificity, an accuracy, an AUC of a ROC, or a combination thereof based on the samples to obtain the plurality of markers.

As used herein, recursive feature elimination (RFE) is one of the feature selection methods that fits a model and removes the least important feature (or features) until the specified number of features is reached.

In some examples, the training set combined with validation (such as, k-fold cross validation (k refers to the number of groups that a given data sample is to be split into), holdout validation, or leave one out cross validation). Hyper-parameter tuning was dependent on type of machine learning model used. Feature reduction may also be performed.

In some embodiments, the present disclosure provides a method of establishing cancer screening module, including obtaining a plurality of initial markers from the plurality of mass spectral data by an initial feature selection method, and then inputting the plurality of initial markers into the machine learning algorithm, wherein the initial feature selection method comprises filter method.

In some examples, filter method: evaluating whether each feature has a statistical relationship (for example, the feature has low variance, there exists correlation between features, there is a difference in the average content of features between groups, etc., to perform feature selection or removal). The known biological or chemical knowledge can be used for screening (for example, the feature has been reported to be related to cancer, the precise molecular weight of the feature is compound that is not produced by human body, such as polymers, surgical supplies (anesthetics, marking pens, ultrasonic conductive glue, etc.).

In some examples, the feature selection method comprises wrapper method or embedded method, in which the wrapper method: using different feature combinations to find the best feature combination for the final model. For example, forward selection, backward selection, exhaustive feature selection, recursive feature elimination, etc.

In some examples, embedded method: feature selection and model training are performed simultaneously with the machine learning algorithm. Such as LASSO regression (reducing the number of features based on the penalty term), random forest (selecting features based on the feature importance ranking of the learning model), etc.

In some embodiments, the present disclosure provides a method of establishing cancer screening module, including using the plurality of markers and the plurality of cancer statuses to establish the cancer screening model by the machine learning algorithm.

### 5. Validate/Evaluate Model

Testing and/or external validation sets evaluates for model overfitting/decrease in performance etc. can be used, and the validation process can be repeated. For example, after using the whole dataset for the first model construction, data input was limited to a specific m/z range to see if performance can be improved. Adding/removing/modifying parameters within the steps of data pre-processing methods are also an option. The verified data (validation set or testing set) can be included in the training set to increase the N value, retrain the model, improve the robustness of the machine, and re-verify with a new set of data.

The above five steps for establishing the machine learning model can be used, not used, or used in any order according to the needs.

### Paper Spray Ionization Platform

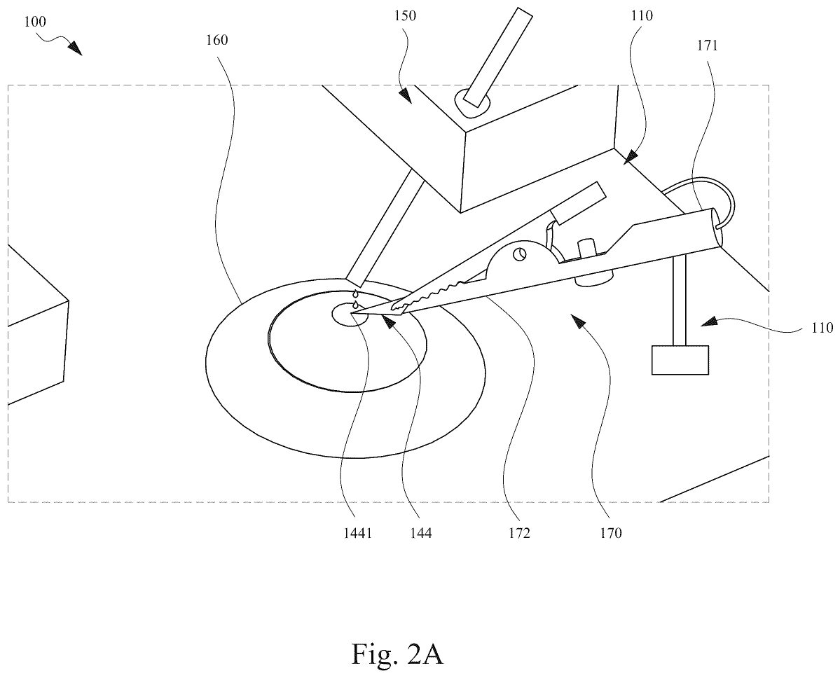

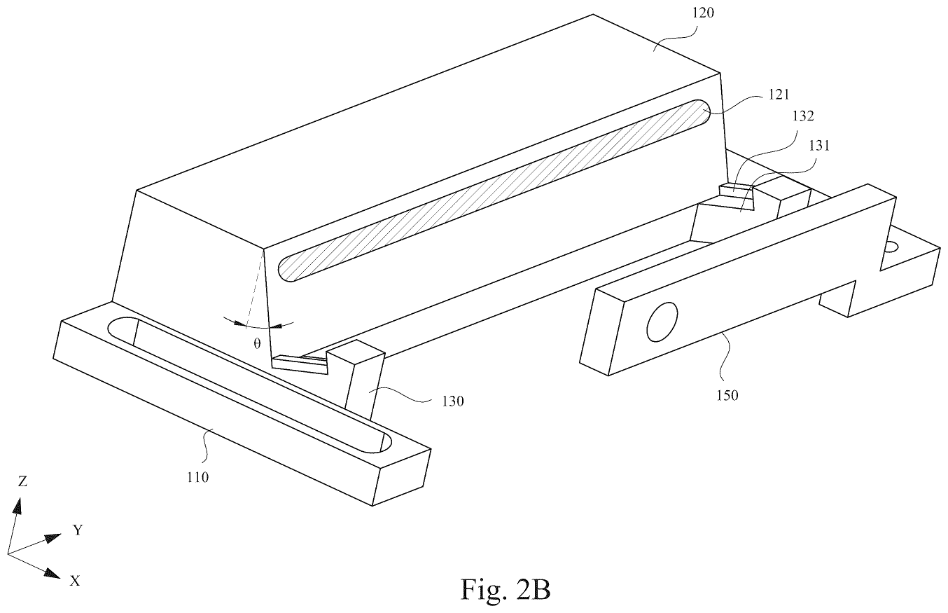

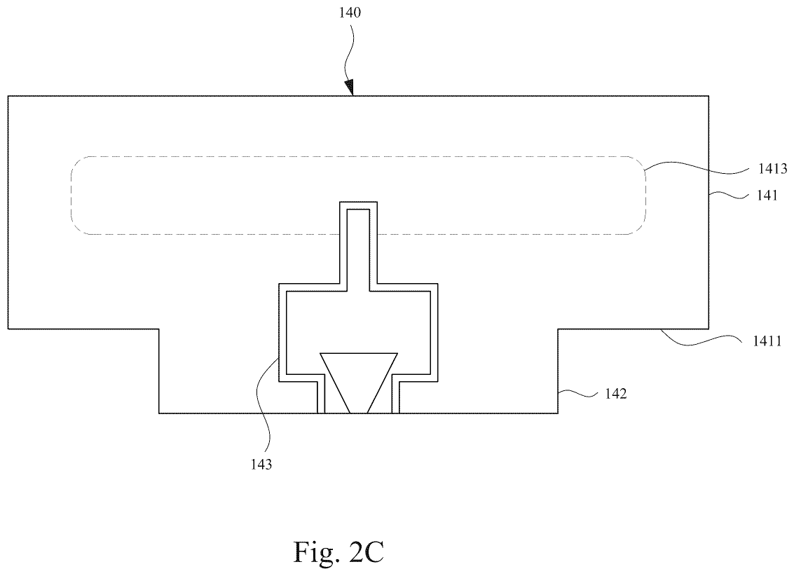

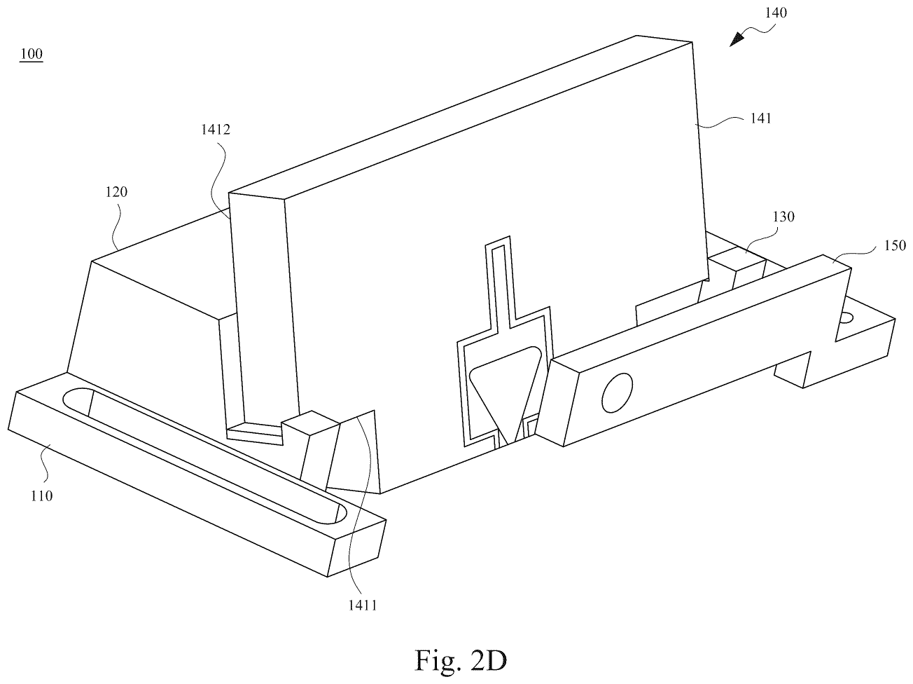

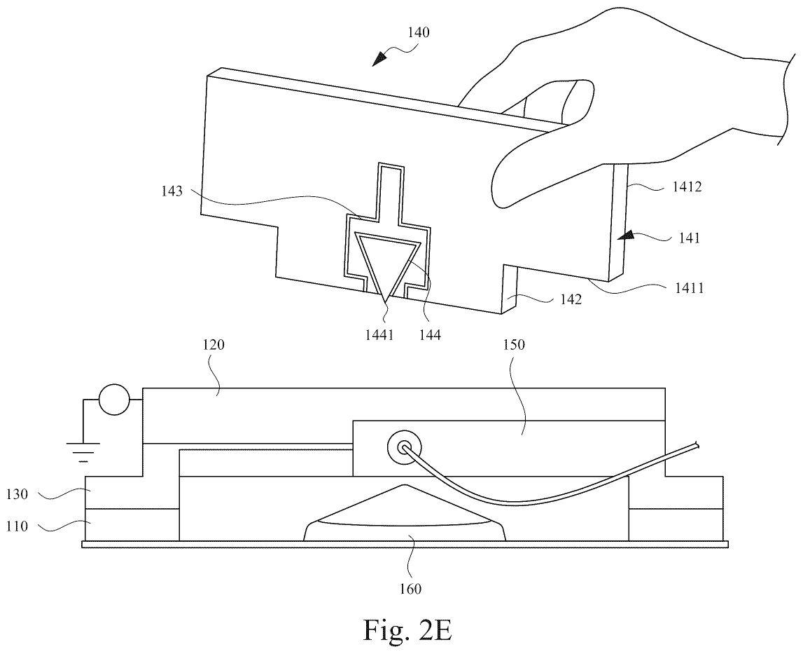

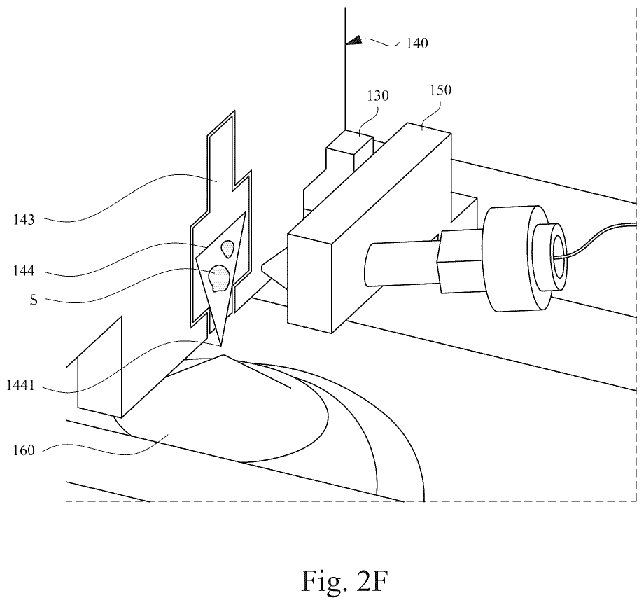

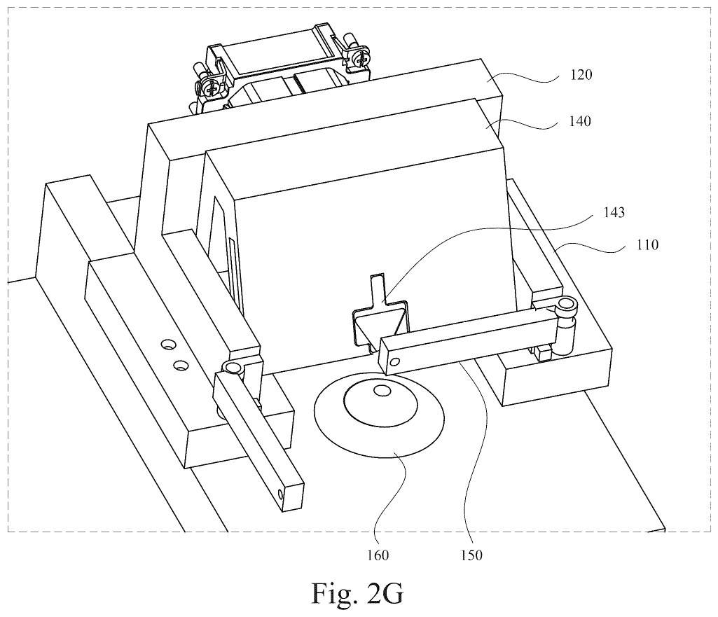

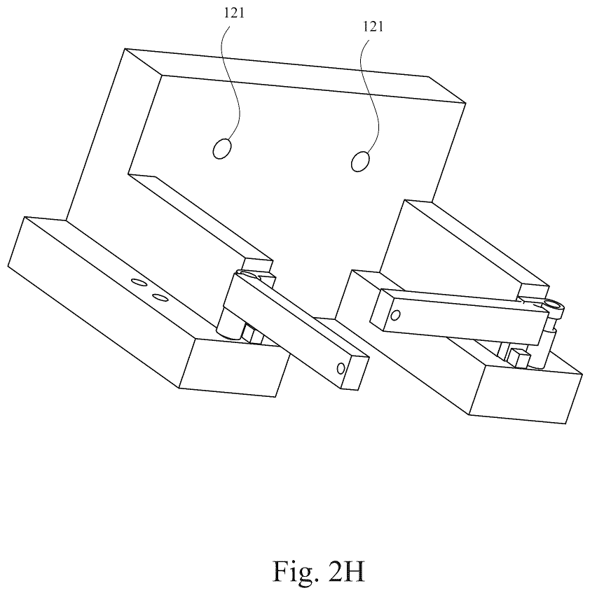

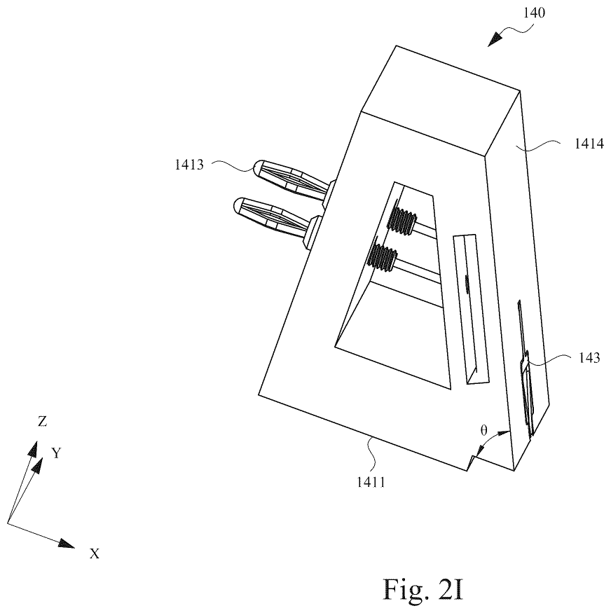

Please refer to FIG. 2A, FIG. 2A is a schematic view of a simply constructed PSI device according to some embodiments of the present disclosure. In some embodiments, the present disclosure provides a PSI device 100, including a base 110, a solvent rack 150, and a clamp 170. The solvent rack 150 is disposed on the base 110. The clamp 170 has a fixing end 171 and a clamping end 172, the fixing end 171 disposed on the base110, the clamping end 172 clamping a paper sheet 144. Mass spectrometry inlet 160, which a portion of a mass spectrometry, collects a plurality of ionized substances ionized from the paper sheet 144.

In some examples, the PSI method includes using the PSI device100, the solvent was placed to the solvent rack 150 of the PSI device 100. Then, the sample was placed on the paper sheet 144 of clamp 170 of the PSI device 100 (in some examples, the paper sheet 144 includes, but is not limited to a filter paper; the sample can be placed on the cut filter paper, or the sample can be placed on, but is not limited to, a filter paper and then cut), and performing the PSI with the solvent to obtain a plurality of ionized substances. And then, the mass spectrometry inlet 160 of the low-resolution mass spectrometer was located below a corner 1441 of the paper sheet 144, the plurality of ionized substances were collected by the mass spectrometry inlet 160, and the samples (the plurality of ionized substances) were analyzed by the low-resolution mass spectrometer. In some examples, the shape of the paper sheet 144 includes, but is not limited to triangle, square etc., as long as there is a portion, which is wide at the top and narrow at the bottom, can be used to place specimen S.

Please refer to FIGS. 2B-2F, FIGS. 2B to 2F are schematic views of the PSI device according to some embodiments of the present disclosure. In some embodiments, the present disclosure provides a PSI device 100, including a base 110, an abutting member 120, a support frame 130, a loading plate 140, and a solvent rack 150. All the above elements can be generated using, but is not limited to, 3D printing technology. The abutting member 120 is disposed on the base 110 and has a first conductive area 121 providing a voltage from 1.5 kV to 5 kV.

Support frame 130 is disposed on the base 110 and extended from a side of the abutting member 120. Specifically, the support frame 130 having two portions respectively located and protruding from two ends of the abutting member 120 at the same side. In some examples, the base 110, the abutting member 120, the support frame 130 are formed in one piece, support frame 130 protrudes from a front end of the abutting member 120, the base 110 extends outward (left and right) from two sides of the abutting member 120. In some examples, the support frame 130 includes two bodies 131 and two stoppers 132 respectively disposed on the two bodies. When the loading plate 140 is placed on the two bodies 131, the two stoppers 132 serves to fix the position of the loading plate 140. In another embodiments, the loading plate 140 abuts against the abutting member 120 and is placed on the base 110 without the support frame 130, the same effect can also be achieved.

The loading plate 140 can be movably placed on the support frame 130, the loading plate 140 includes a body 141, a protrusion 142, and a metal placing piece 143. The body 141 has a bottom surface 1411 adjacent to a side surface 1412 of the bottom surface 1411, the side surface 1412 can movably abut against the abutting member 120, and the bottom surface 1411 can movably abut against the base110; in some examples, the side surface 1412 having a second conductive area 1413 is electrically connected to the first conductive area 121 of the abutting member 120 when in contact. The protrusion 142 protrudes outward from the bottom surface1411 of the body 141; in some examples, the protrusion 142 and the bodyl41 are formed in one piece. The metal placing piece 143 is disposed on the body 141 and the protrusion 142, and is metal with conductive material, and a back of the metal placing piece 143 is electrically connected to a power supply. The paper sheet 144 is placed on the metal placing piece143, a corner 1441 of the paper sheet 144 protrudes outward from the protrusion 142, and a protruding direction of the corner 1441 and a protruding direction of the protrusion 142 are the same and face toward base110; In some examples, the shape of the paper sheet 144 includes, but is not limited to triangle, square etc., as long as there is a portion, which is wide at the top and narrow at the bottom, can be used to place specimen S; in some examples, the paper sheet 144 includes, but is not limited to a filter paper. In the three-dimensional space of the X-axis, Y-axis, and Z-axis, when the loading plate 140 of the present disclosure abut against the abutting member 120, it has a specific angle θ compared to the Z-axis, such as 0 degree to 90 degrees, so that it's easy for people who are not familiar with mass spectrometers to use during operation, and can obtain the same mass spectrum results as professionals.

The solvent rack 150 is disposed on the base 110.

In some examples, as shown in FIGS. 2C to 2E, the PSI method includes using the PSI device100, the solvent was placed to the solvent rack 150 of the PSI device 100. Then, the sample was placed on the paper sheet 144 of the loading plate 140 of the PSI device 100 (the sample can be placed on the cut filter paper, or the sample can be placed on the filter paper and then cut), and PSI is performed with the solvent to obtain a plurality of ionized substances. And then, the mass spectrometry inlet 160 of the low-resolution mass spectrometer was located below a corner 1441 of the paper sheet 144, the plurality of ionized substances were collected by the mass spectrometry inlet 160, and the samples (the plurality of ionized substances) were analyzed by the low-resolution mass spectrometer.

FIGS. 2G to 2I are schematic views of a PSI device according to another embodiment of the present disclosure. Similar to the embodiment of FIGS. 2B to 2F, the present embodiment provides a PSI device100, including a base 110, an abutting member 120, a loading plate 140, and a solvent rack 150. The differences are that the abutting member 120 is a vertical cube disposed on the base 110, and the first conductive area 121 presents a plurality of dot-shaped contacts (such as banana sockets), providing a voltage of 1.5 kV to 5 kV. Next, a side surface of the loading plate 140 is trapezoidal, and the second conductive area 1413 (such as banana plugs) is electrically connected to the metal placing piece 143 via a wire (such as a pogo pin). In the three-dimensional space of the X-axis, Y-axis, and Z-axis, there is a specific angle θ between the slope 1414 of the loading plate 140 and the bottom surface 1411 of the present disclosure, such as 0 degree to 90 degrees, so that it's easy for people who are not familiar with mass spectrometers to use during operation, and can obtain the same mass spectrum results as professionals. Then, the solvent rack 150 is pivotally connected to the base 110, so that the solvent rack 150 can movably adjust its position. The remaining element and use methods are similar to FIGS. 2B to 2F, and will not be described again herein.

PSI and mass spectrometer analysis can be applied to common specimens for screening, such as surgical tissue, lymph node tissue, fine needle biopsy and CNB, tissue smears, biological fluids (such as blood, plasma, urine, etc.). The operator only needs to place the sample to be tested on the filter paper, cut it and put it into the metal placing piece, and place the loading plate on the support frame of the platform. The mass spectrum can be collected within the next two minutes.

Various samples analyzable with designed paper spray ionization platform of present disclosure without noticeable change in m/z profiles for the same sample. Specificity, 3D printed platform was tested by personnel of 3 different backgrounds: 1. proficient with paper spray ionization mass spectrometry (PSI proficient); 2. having experience handling mass spectrometer of other ionization sources; 3. with no prior background in mass spectrometry. There was no observable difference in the mass spectrum m/z profiles acquired by personnel of different background for the same sample (data not shown).

As used herein, “m/z” is “m/z bin.” Since the present disclosure uses a low-resolution mass spectrometer, m/z bins are used for modeling. For example, using 1 Dalton (Da) for m/z binning, m/z bin 700.5 contains compounds from m/z 700-701, or compounds of m/z 700.5-701.5 depending on the start m/z of the bin. There may be one or more compounds in this bin, and one or more combinations of the one or more compounds may be used as a basis for identifying cancer types. If there are a plurality of compounds, for example, when the exact masses are 700.5678, 700.6375 and 700.7345, and the content of 700.5678 is higher than the other two, the compound 700.5678 with the highest content will be regarded as a putative ID for easy identification, but It does not mean that the compound that is truly used to identify cancer types must be 700.5678. If the putative ID of the compound with the higher content cannot be inferred, the compound with the second higher content will be used as the possible ID, and so on.

### Example 1.1 Identifying Benign Breast Tumor and Malignant Breast Tumor

Molecular analysis of breast CNB using miniature mass spectrometry coupled with paper spray ionization (MiniMaP):

We coupled a home-built PSI device with a miniature single quadrupole mass spectrometer for the in situ analysis of breast CNB samples. To acquire the m/z profiles of the samples, the CNB sample was placed on the filter paper and held by a copper clip on the top of the miniature mass spectrometer inlet (as shown in FIG. 2A, the embodiments of FIGS. 2B to 2I can also achieve the same experimental result). After the application of high voltage and spray solvent, the chemicals (ionized substance) of the CNB were extracted and sprayed into the mass spectrometer to reveal the molecular information. No extra gas, which is necessary for common ionization sources for spray nebulization or plasma generation, is required for the PSI MS setup.

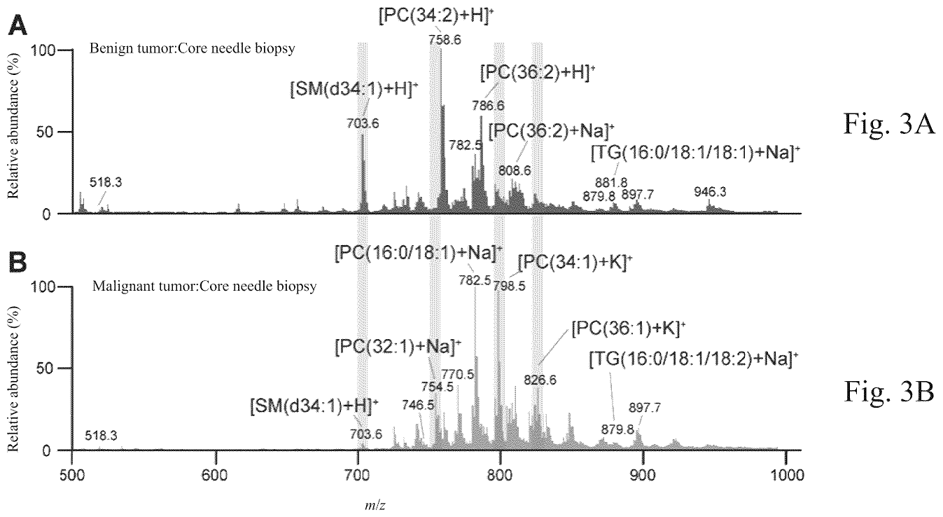

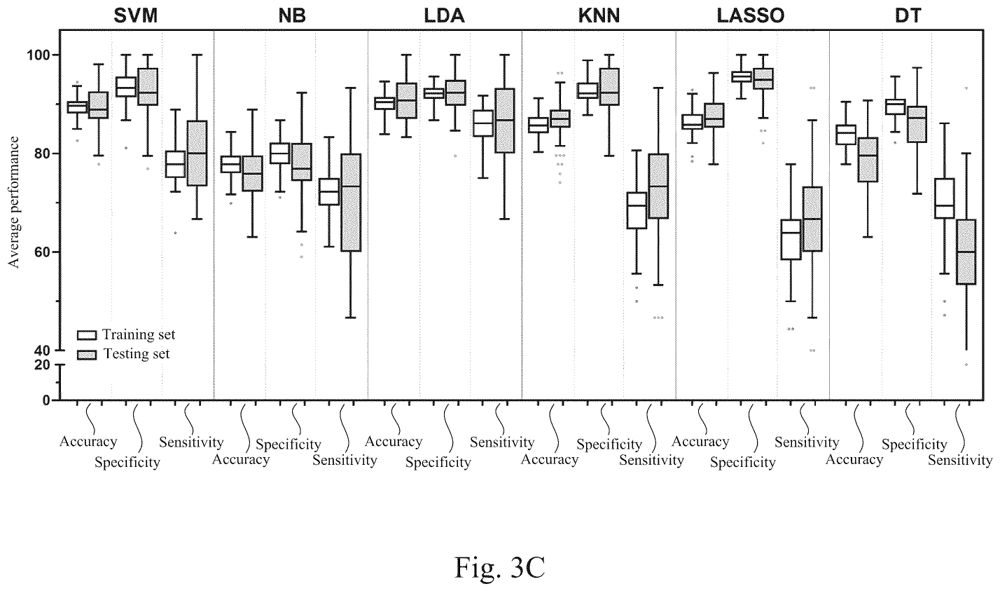

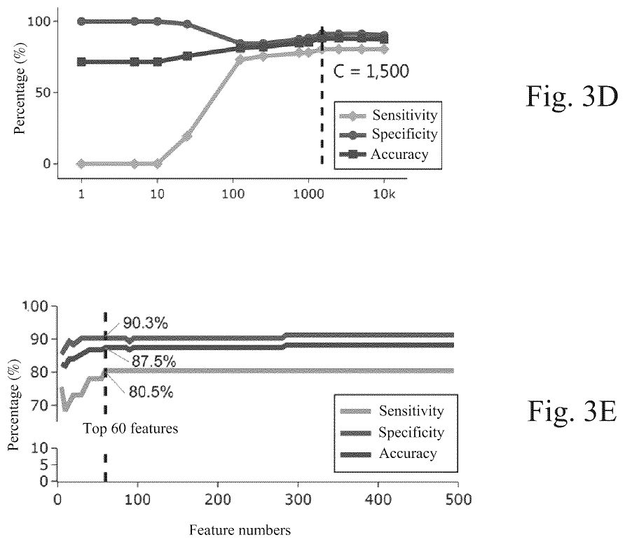

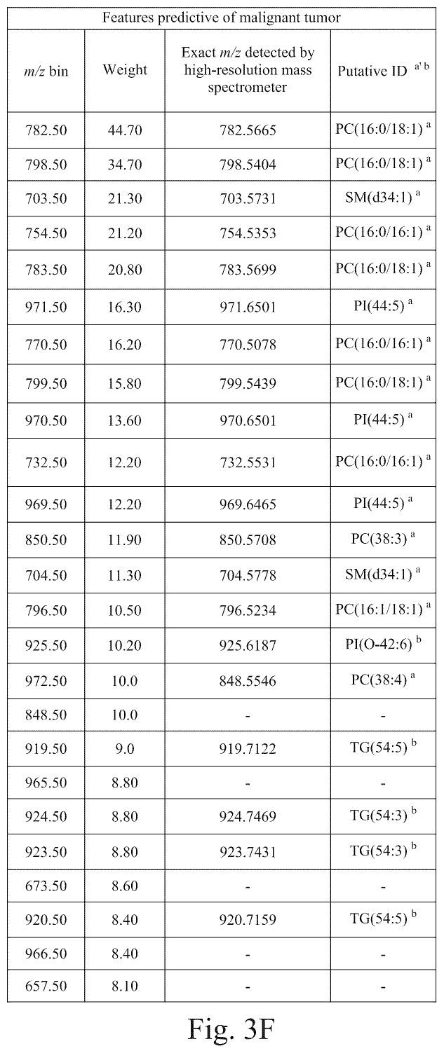

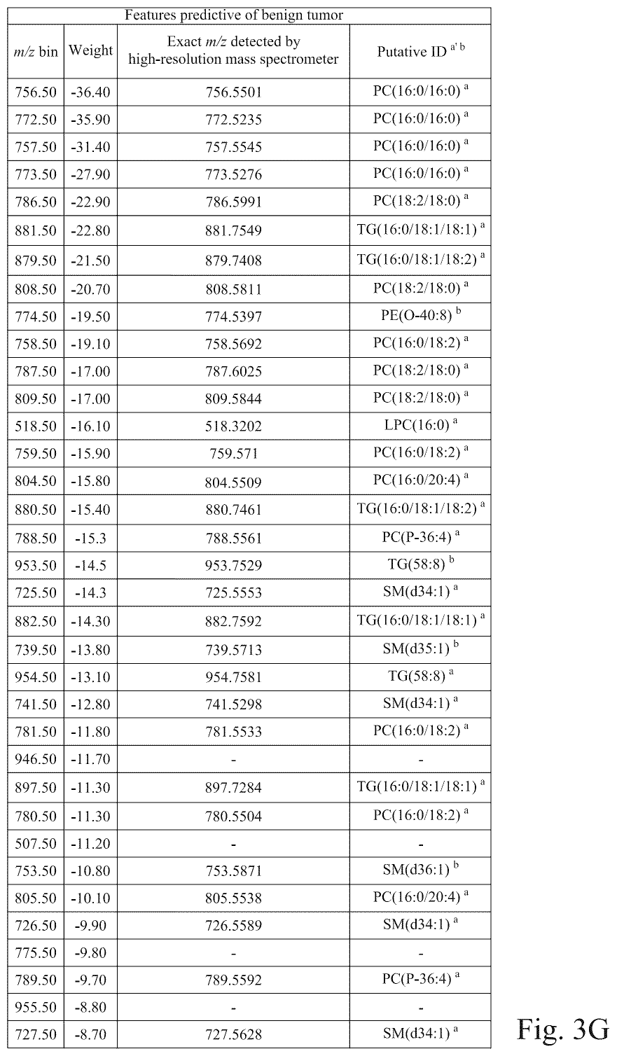

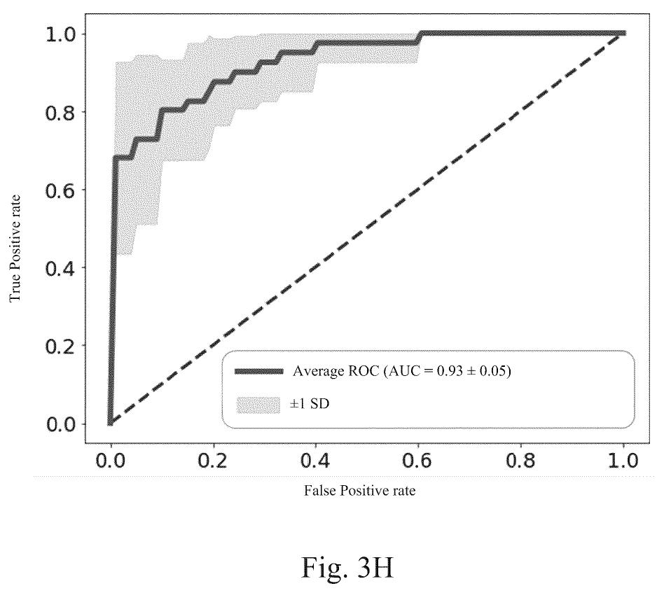

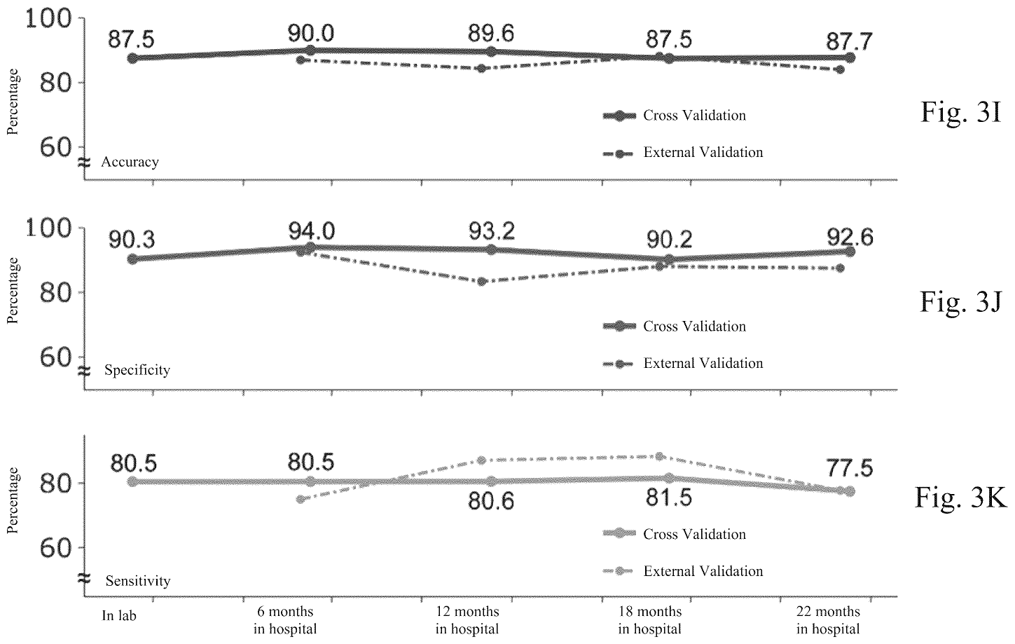

We collected the mass spectra of the biopsies in positive-ion mode with the mass range of m/z 500 to 1000 to investigate the metabolites of CNB. The representative spectra of a benign and malignant biopsy are shown in FIGS. 3A and 3B. The annotated ions in figures were putatively identified using tandem mass spectrometry (MS/MS) on a high-resolution MS using the same PSI setup. Commonly observed lipid species, including triacylglycerol (TG), sphingomyelin (SM), glycerophosphocholine (PC), and glycerophosphoethanolamine (PE), were detected.

To test the ionization stability of the PSI interface on the miniature mass spectrometer, we continuously analyzed a specimen for 15 minutes. The total ion chromatogram (TIC) indicates that during the first 30 seconds, metabolites in the biopsy sample were gradually extracted by the spray solvent and ionized at the paper tip. The MS profile and intensity of TIC were stabilized after 30 sec. The signal started to fluctuate after 13 minutes because of the over-accumulation of solvent on the filter paper, causing the electric arcs appear between the paper tip and MS inlet. This result demonstrated that stable lipid signals can be acquired from CNB using the MiniMaP for at least 10 minutes. Compared to the time frame of our experiment, which we collected the mass spectra for 30 seconds after the signal stabilized, the MiniMaP provided sufficient time for acquiring screening MS spectra.

### Choosing Different Machine Learning Algorithm

FIG. 3C shows expressions of benign and malignant breast tumor biopsy analyzed by machine learning algorithm according to some embodiments of the present disclosure. Data collected using CNB (N=180; benign=129, malignant=51) with 6 different algorithms (SVM, Naive Bayesian (NB), linear discriminant analysis (LDA), KNN, decision tree (DT)) to obtain prediction results (hyper-parameters had been adjusted). Training set and validation set were randomly split 100 times, and 10-fold cross validation was used to evaluate the screening effects of different machine learning algorithms. Adjusting hyper-parameters was a necessary process to evaluate the capabilities of the model, so the prediction results using the default parameters were not used in the end. The default parameters were parameters that can usually achieve good prediction results when using the model to build different pieces of data. However, if the default parameters are simply executed, an algorithm that is not suitable for the data may accidentally select. SVM and LDA perform great among the algorithms as shown in the figure. LDA is a common linear method for feature extraction, in which the initial feature set was reduced to a set of features in a low-dimensional space, resulting in new axis (or multiple axes) to maximize the distance between the means of two categories, and to maximally reduce variation within each category. Although the accuracy of LDA is relatively high, the general specificity is high during SVM training, so this model was finally selected to increase the possibility of correct prediction of benign tumor and to reduce false positives (to avoid overtreatment).

### Using Machine Learning Algorithm

We established a multivariate statistical classification model with support vector machine recursive feature elimination (SVM-RFE), a machine learning algorithm, to differentiate the m/z profile between benign and malignant CNBs. First, the 180 CNBs were randomly split into training and testing set with a ratio of 8 to 2. That is, mass spectral data were obtained from 180 samples by the aforementioned MiniMaP, and then 129 benign tumors were split into the first training set and the first test set with a ratio of 8 to 2 and 51 malignant tumors were split into the second training set and the second test set with a ratio of 8 to 2. Training set (i.e. the first training set and the second training set) was trained with 5-fold cross validation to tune optimal hyper-parameters (FIGS. 3D and 3E). Linear SVM was first used to calculate the mathematical weights of each feature. The features were then ranked by their importance and removed successively by SVM-RFE to estimate the performance from remaining features (FIG. 3E). Through this algorithm, fewer features are extracted from the original input, thus avoiding overfitting and simplifying the results to improve interpretability. The prediction accuracy and number of significant metabolites were considered to select an optimal discriminant classifier. Ultimately, 60 features were retained in the SVM-RFE model for breast cancer identification (FIGS. 3F and 3G). These 60 features are 60 markers.

By considering multiple markers, the discriminant performance was improved compared to using single molecular ion alone. The optimized model achieved an averaged area under ROC curve of 0.93 (FIG. 3H). The sensitivity of 80.5%, specificity of 90.3%, and an overall accuracy of 87.5% were achieved in training set (Table 1). The results are comparable to a previous research using PSI on a high resolution mass spectrometer for breast cancer diagnosis (accuracy=87.5%, n=159). We further applied the model to the testing set. The model performed a sensitivity, specificity, and overall accuracy of 80.0%, 92.3%, and 88.9%, respectively. The performance of the model on training set and testing set was consistent, showing that the over-fitting issue was minimized in our SVM-RFE model.

TABLE 1

| | Pathological | | | | |
|---|---|---|---|---|---|
| | diagnosis | n patients | Sensitivity^{[a]} | Specificity^{[b]} | Accuracy^{[c]} |
| Training set | Benign | 103 | 80.5% | 90.3% | 87.5% |
| (5-fold cross | Malignant | 41 | (33/41) | (93/103) | (126/144) |
| validation) | | | | | |
| Testing set | Benign | 26 | 80.0% | 92.3% | 88.9% |
| | Malignant | 10 | (8/10) | (24/26) | (32/36) |
| Validation | Benign | 359 | 83.0% | 85.0% | 84.4% |
| set (Analyzed | Malignant | 153 | (127/153) | (305/359) | (432/512) |
| in hospital) | Ductal | 28 | 78.6% | — | 78.6% |
| | carcinoma | | (22/28) | | (22/28) |
| | in situ | | | | |

^{[a]}Sensitivity = [TP/(TP + FN)],
^{[b]}Specificity = [TN/(TN + FP)],
^{[c]}Accuracy = [(TP + TN)/(TP + TN + FP + FN)]
TP = true positive;
TN = true negative;
FP = false positive;
FN = false negative

With the SVM-RFE algorithm, the ions with higher screening power were selected, assisting the discovery of potential breast cancer biomarkers. Among hundreds of molecular features detected by the MiniMaP, our model selected 60 for breast cancer diagnosis (FIGS. 3F and 3G). The features that show higher abundance in malignant tissue were assigned positive weightings, whereas features that are more abundant in benign tissue were assigned negative weightings. To further reveal the molecular differences between benign and malignant tumor, we identify the predictive metabolites selected by the SVM-RFE model using PSI high-resolution tandem MS. In our model, glycerophosphocholine (PC) species play significant roles for classification. Ion with m/z 782.5, identified as PC (16:0/18:1), was assigned the highest weight toward malignant tumor, whereas m/z 756.5, identified as PC (16:0/16:0), was assigned the highest weight toward benign tumor. Our platform provides not only screening results but also the molecular information from the CNB. The accumulated molecular information could be further interpreted to assist the understanding of breast cancer and the achievement of precision medicine.

### User-Friendly Platform Development

The integration of PSI and MMS (miniature mass spectrometer) allows the tissue to be analyzed with minimal sample pretreatments, which greatly reduces the difficulties for end users in hospitals to operate the instrument. Besides MS analysis procedure, the simplification of MS data processing and reporting process is also critical to transferring the screening platform into clinics. Therefore, we designed an easy-to-use graphical-user-interface (GUI) to assist end users without professional programming backgrounds to accomplish tissue assessment. The GUI included the data preprocessing pipeline and optimized machine learning model. Once the query MS spectrum is loaded into the GUI, the screening results based on the MS profile will be presented with just one click. Overall, through the integration of PSI, a MMS, and GUI, we demonstrated MiniMaP as a user-friendly platform for medical professionals to determine the tumor type of breast CNB samples. With the simplified analysis protocol and trained screening model, we transferred the platform into clinics for validation.

### Multicenter Validation of the MiniMaP Platform in Hospital

After using the analytical platform in the hospital, a total of 540 biopsy samples were collected, including 359 benign, 181 malignant tumor biopsies. The on-site screening can be accomplished within 5 min upon acquiring each sample. After comparison with the pathological reports, our model achieved accuracy, sensitivity, and specificity of 84.4%, 83.0%, and 85.0%, respectively (Table 1 as above mentioned). The specificity, sensitivity and overall accuracy were similar to in-lab analysis. The comparable screening performance indicated the stability of our platform and the robustness of our statistical classifier. Through the multicenter study, we demonstrated that the MiniMaP platform can provide rapid on-site breast cancer screening in hospitals, which shows great potential to be incorporated into routine clinical procedures.

### Continual Learning of the Screening Model

With the accumulation of biopsies collected in hospital, we were able to continuously update the screening model with increasing amount of data. The process of continual learning allows the model to incrementally learn and achieve strengthened performance. After transferring the MiniMaP platform to hospital, we retrained the screening model every six months. The newly collected samples were included into training set for model optimization. The 5-fold cross validation results of the retrained models were shown in FIGS. 3I, 3J, and 3K. The retrained models were then evaluated with the samples collected in the next six months.

During the 22 months, we updated the screening model four times. The final model, trained by 684 samples, reached an overall accuracy of 87.7%. The screening accuracy was improved after retraining, and the model became more robust with increasing number of training data. In addition, compared to the performance of directly applying the initial model to all the data acquired in the hospital, the specificity was increased from 85% to above 90%. With the application of continual learning, we demonstrated that the screening model can continuously learn from new data, improving the robustness of the MiniMaP platform in breast cancer screening.

The treatment method of the benign breast tumor includes, but is not limited to clinical examinations, radiological examination, histological examination, surgery, or a combination thereof. The treatment method of the malignant breast tumor includes, but is not limited to surgery, radiation therapy, chemotherapy, hormone therapy, targeted therapy, immunotherapy, or a combination thereof.

### Example 1.1.1 Using Different Numbers of Breast Cancer Biomarkers to Distinguish Benign Breast Tumor and Malignant Breast Tumor

As shown in Table 1, 60 biomarkers as shown in FIGS. 3F and 3G in 144 samples (41 malignant tumor, 103 benign tumor) were used to obtain 87.5% accuracy, 80.5% sensitivity, and 90.3% specificity. The experimental method of this example is almost the same as that of Example 1.1, the difference is that 11 biomarkers (m/z 782.5, m/z 798.5, m/z 754.5, m/z 770.5, m/z 923.5, m/z 772.5, m/z 757.5, m/z 774.5, m/z 788.5, m/z 753.5, and m/z 727.5) of 60 biomarkers as shown in FIGS. 3F and 3G were used to obtain 86.8% accuracy, 73.2% sensitivity, and 92.2% specificity.

Even if the number of samples increases to 652 (224 malignant tumor, 428 benign tumor), the above 11 biomarkers can still obtain an accuracy of 83.4% accuracy, 75.9% sensitivity, and 87.4% specificity.

### Example 1.2 Breast Cancer Molecular Subtype

In addition to diagnosis, the determination of molecular subtype is also critical in breast cancer since the medical treatments vary among different subtypes. The molecular subtypes of breast cancer are defined by the genetic expression level of the hormone receptor (HR) and human epidermal growth factor receptor 2 (HER2). Luminal-like subtype is characterized by high expression of HR, which includes expression of estrogen receptor (ER) and/or progesterone receptor (PR). The subtype of HER2 characterized in high level of HER2 and low level of HR expression. In the case of triple-negative breast cancer, the expression of ER, PR, and HER2 are low. Currently, the immunohistochemistry (IHC) assays and fluorescence in situ hybridization (FISH) assays are used in clinics to determine the status of HR and HER2, respectively. However, the IHC and FISH assays are time-consuming and may present subjectivity in data interpretation. A rapid and precise breast cancer subtyping technique is desirable to assist physicians in deciding medical treatment and improve patient care.

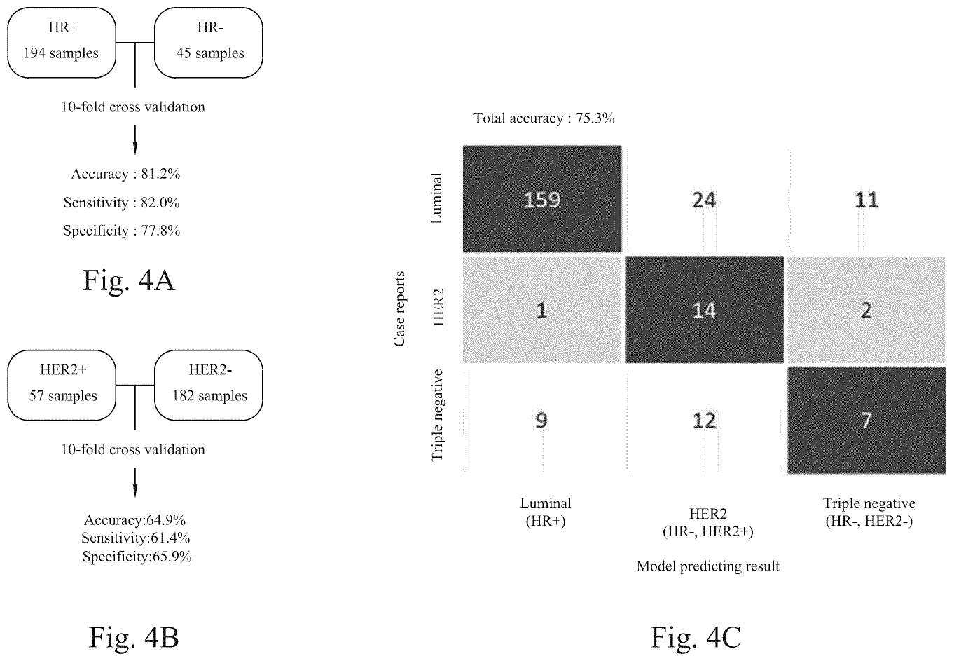

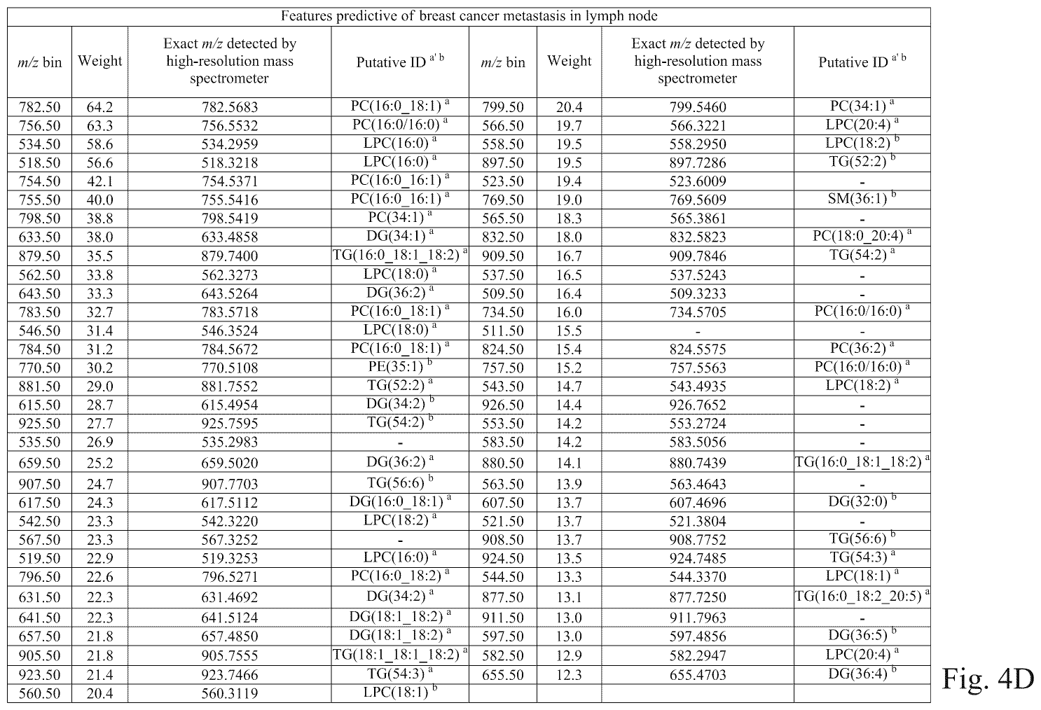

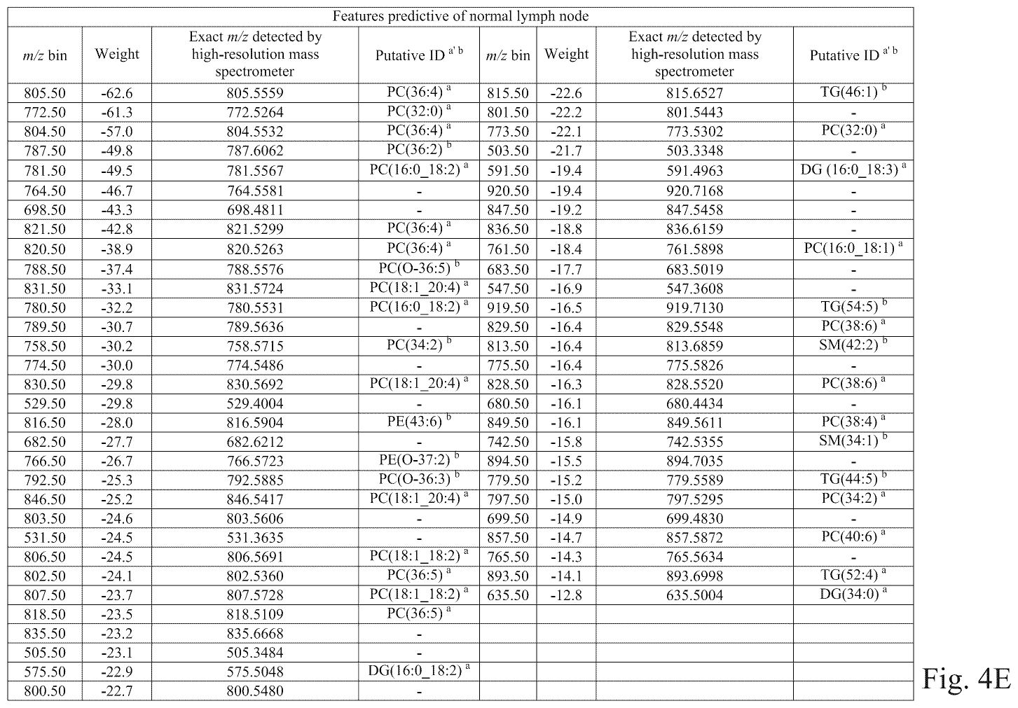

With the increasing number of malignant biopsies collected in the hospital, we attempted to construct breast cancer subtyping models according to the lipid profile acquired using MiniMaP. First, we trained two SVM-RFE models to differentiate the status of HR and HER2, respectively. Ten-fold cross validation was applied to tune the optimal hyper-parameters. In the case of HR classification, our model achieved accuracy, sensitivity, and specificity of 81.2%, 82.0%, and 77.8%, respectively (FIG. 4A). In contrast, the accuracy of 64.9%, sensitivity of 61.4%, and specificity of 65.9% were achieved in the determination of HER2 status (FIG. 4B). Next, we determined the molecular subtype of each patient according to the predicted HR and HER2 statuses (FIG. 4C). By comparing to the pathological reports, our models achieved an overall accuracy of 75.3% in predicting breast cancer molecular subtype. This result presented a proof-of-concept that the molecular subtyping can be rapidly determined with our MiniMaP platform.

The treatment method of HR subtype of breast cancer includes, but is not limited to hormone therapy, chemotherapy, surgery, radiation therapy, or a combination thereof. The treatment method of the HER2 subtype of breast cancer includes, but is not limited to surgery, radiation therapy, targeted therapy, chemotherapy, or a combination thereof. The treatment method of the triple negative breast cancer (TNBC) subtype includes, but is not limited to surgery, radiation therapy, chemotherapy, immunotherapy, or a combination thereof.

### Example 1.3 Identifying Whether Breast Cancer Tumor Metastasis in Lymph Node

One or more of the operations shown in Example 1.3 are the same as or similar to those explained with respect to Example 1.1, and the detailed explanation may be omitted. The differences between Example 1.3 and Example 1.1 are that specimens were obtained from normal lymph node and lymph nodes with breast cancer cell metastasis. Please refer to Table 2 below for detailed amount of specimen. This example is lymph node tissue removed during surgery, the tissue was bisected, and the bisected faces of the specimen was smeared on filter paper. The MiniMaP platform was also used in the example to obtain the mass spectrum, and mass spectra from biopsies were collected in positive ion mode in the mass range of m/z 500 to 1000. Then the training set of the example was trained through 10-fold cross validation, and C was set to 2,380 for further optimization of SVM-RFE to adjust the optimal hyper-parameters to distinguish breast cancer without tumor metastasis in lymph node tissue and breast cancer with tumor metastasis in lymph node tissue from mass spectra. Finally, 122 features were retained in the SVM-RFE model (FIGS. 4D to 4E).

The result shows that the sensitivity of the training set was 77.8%, the specificity was 90.8%, and the accuracy was 88.8% (Table 2).

The treatment method for breast cancer with tumor metastasis in lymph node tissue include, but is not limited to, removal of axillary lymph glands, surgery, radiotherapy, chemotherapy, hormone therapy, targeted therapy, immunotherapy, or a combination thereof.

In addition, 26 features (m/z 509.5, m/z 531.5, m/z 534.5, m/z 567.5, m/z 575.5, m/z 615.5, m/z 641.5, m/z 643.5, m/z 698.5, m/z 742.5, m/z 754.5, m/z 758.5, m/z 761.5, m/z 781.5, m/z 782.5, m/z 797.5, m/z 798.5, m/z 805.5, m/z 820.5, m/z 824.5, m/z 828.5, m/z 829.5, m/z 830.5, m/z 846.5, m/z 879.5, and m/z 880.5) were used for modeling under the same experimental method, the sensitivity of the training set was 73.5%, the specificity was 96.5%, and the accuracy was 92.1%.

### Example 1.4 Identifying Breast Cancer Tissue and Normal Breast Tissue at Surgical Margin

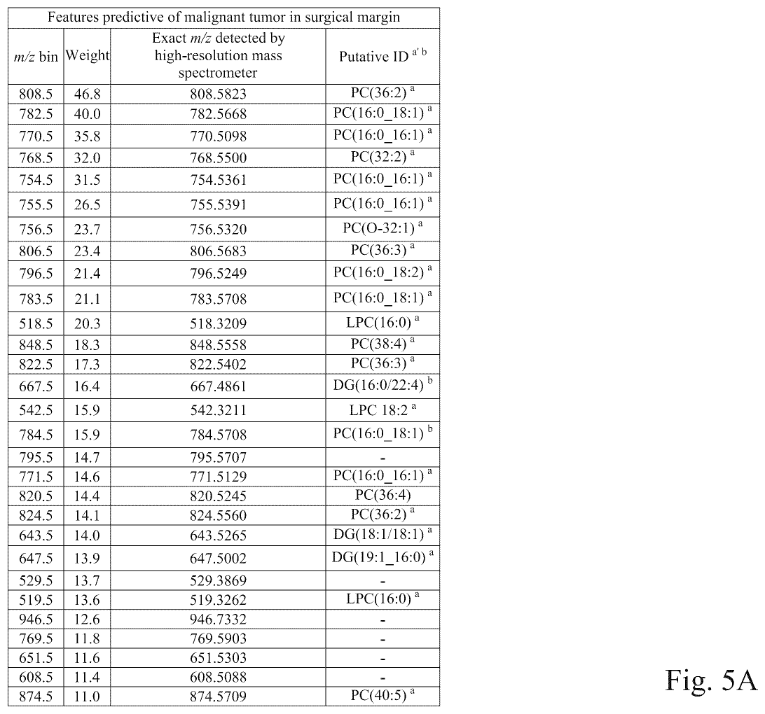

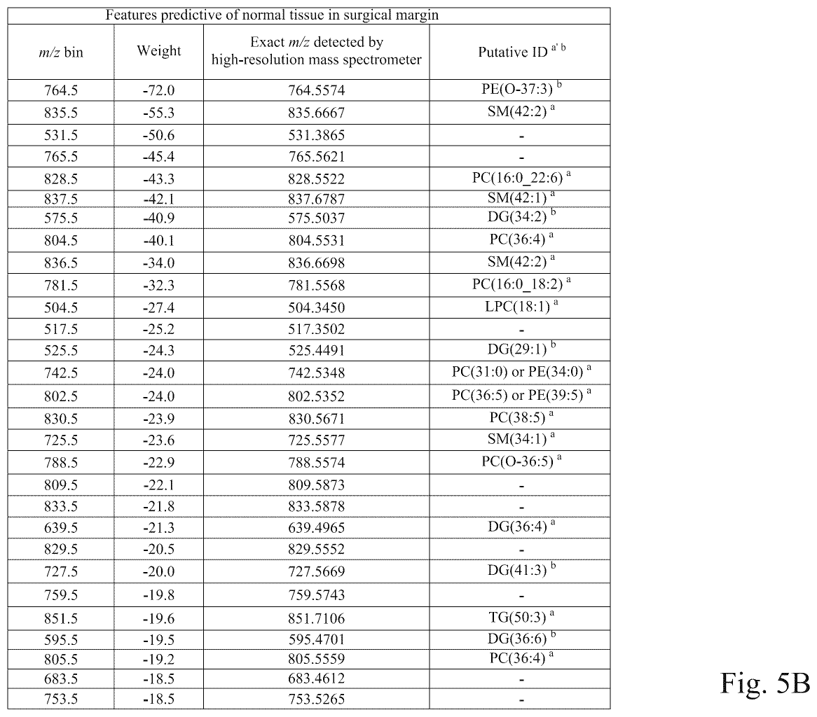

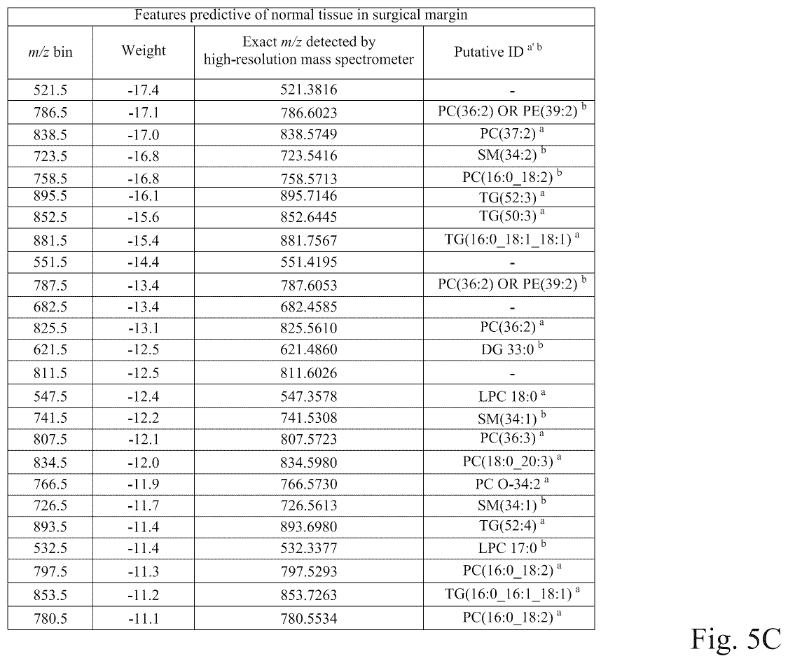

One or more of the operations shown in Example 1.4 are the same as or similar to those explained with respect to Example 1.1, and the detailed explanation may be omitted. The differences between Example 1.4 and Example 1.1 are that specimens were obtained from normal breast tissue (or normal resection margin tissues), and breast cancer tissue. Please refer to Table 2 below for detailed amount of specimen. This example is that the filter paper was dabbed/smeared on specimen (smear, or called tissue-stained filter paper). The MiniMaP platform was also used in the example to obtain the mass spectrum, and mass spectra from biopsies were collected in positive ion mode in the mass range of m/z 500 to 1000. Then the training set of the example was trained through 5-fold cross validation, and C was set to 1,800 for further optimization of SVM-RFE to adjust the optimal hyper-parameters to distinguish normal breast tissue and breast cancer tissue from mass spectra. Finally, 83 features were retained in the SVM-RFE model (FIGS. 5A to 5C).

The result shows that the sensitivity of the training set was 86.5%, the specificity was 95.3%, and the accuracy was 91.2% (Table 2).

In addition, 30 features (m/z 504.5, m/z 835.5, m/z 575.5, m/z 723.5, m/z 764.5, m/z 837.5, m/z 804.5, m/z 547.5, m/z 853.5, m/z 765.5, m/z 788.5, m/z 828.5, m/z 781.5, m/z 727.5, m/z 759.5, m/z 836.5, m/z 682.5, m/z 753.5, m/z 786.5, m/z 770.5, m/z 805.5, m/z 518.5, m/z 768.5, m/z 755.5, m/z 782.5, m/z 756.5, m/z 824.5, m/z 754.5, m/z 647.5, and m/z 848.5) were used for modeling under the same experimental method, the sensitivity of the training set was 88.2%, the specificity was 93.5%, and the accuracy was 90.8%

### Example 2.1 Identifying Normal Skin Tissue, Benign Nevus Tissue or Melanoma

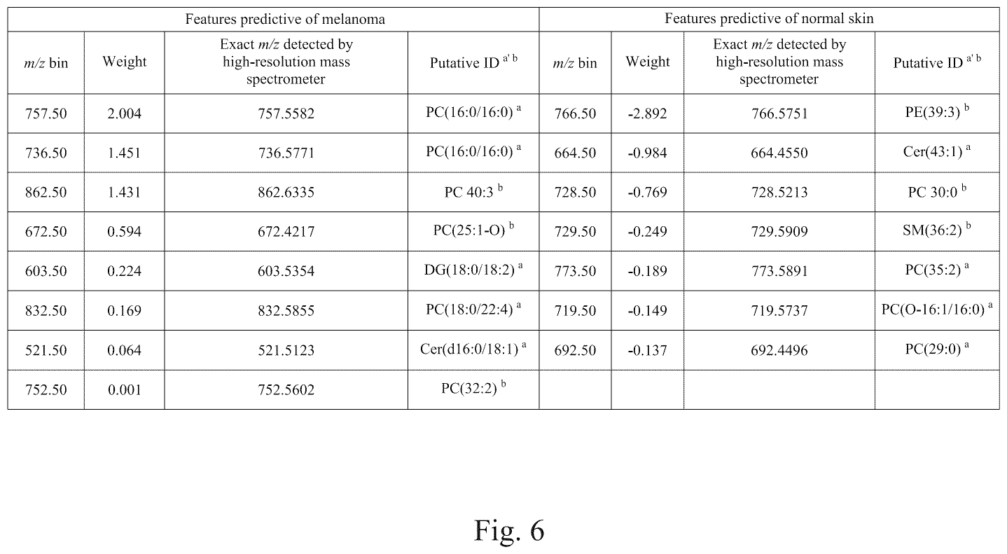

One or more of the operations shown in Example 2.1 are the same as or similar to those explained with respect to Example 1.1, and the detailed explanation may be omitted. The differences between Example 2.1 and Example 1.1 are that specimens were obtained from normal skin tissue, and/or benign nevus tissue, and melanoma; LASSO regression was chosen for the algorithm model. Please refer to Table 2 below for detailed amount of specimen. The specimens of this example were obtained from surgery. The specimens were placed in 150 mM cold ammonium formate for 15 mins, then placed on filter paper. The MiniMaP platform was also used in the example to analyze the mass spectrum, and mass spectra from surgical tissue were collected in positive ion mode in the mass range of m/z 500 to 1000. Then the training set of the example was trained through 10-fold cross validation to distinguish normal skin tissue, benign nevus tissue or melanoma from mass spectra. Finally, 15 features (m/z 757.5, m/z 736.5, m/z 862.5, m/z 672.5, m/z 603.5, m/z 832.5, m/z 521.5, m/z 752.5, m/z 766.5, m/z 664.5, m/z 728.5, m/z 729.5, m/z 773.5, m/z 719.5, and m/z 692.5) were retained in the LASSO model (FIG. 6).

The result shows that the sensitivity of the training set was 87.5%, the specificity was 92.9%, and the accuracy was 91.7% (Table 2).

The treatment method of melanoma includes, but is not limited to surgery, immunotherapy, targeted therapy, radiation therapy, chemotherapy, or a combination thereof.

### Example 2.2 Identifying the Presence of Melanoma Cancer Cells Metastasis in Lymph Node

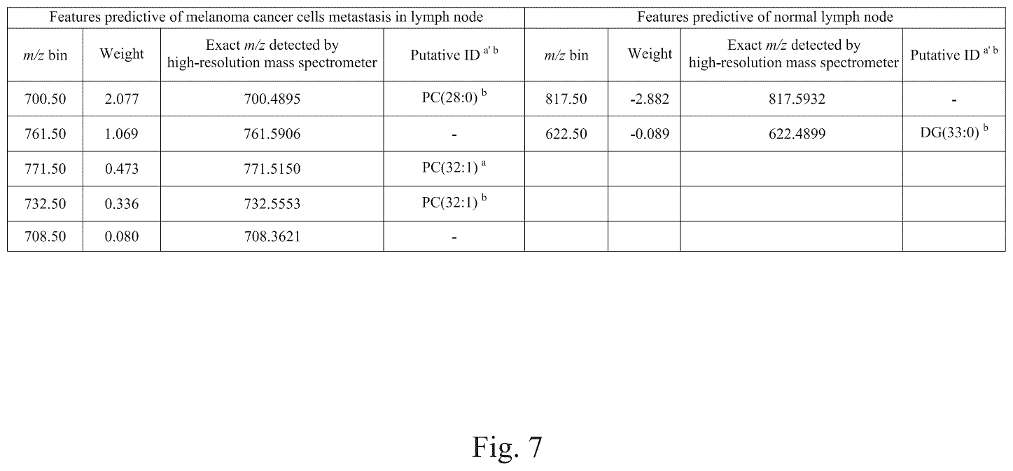

One or more of the operations shown in Example 2.2 are the same as or similar to those explained with respect to Example 1.1, and the detailed explanation may be omitted. The differences between Example 2.2 and Example 1.1 are that specimens were obtained from normal lymph nodes and lymph nodes (LNs) with melanoma cancer cell metastasis; LASSO regression was chosen for the algorithm model. Please refer to Table 2 below for detailed amount of specimen. The lymph node tissues of this example were obtained from surgery. The specimens were placed on filter paper. The MiniMaP platform was also used in the example to analyze the mass spectrum, and mass spectra from surgical tissue were collected in positive ion mode in the mass range of m/z 500 to 1000. Then the training set of the example was trained through 5-fold cross validation to distinguish normal lymph nodes and lymph nodes with melanoma cancer cell metastasis. Finally, 7 features (m/z 700.5, m/z 761.5, m/z 771.5, m/z 732.5, m/z 708.5, m/z 817.5, and m/z 622.5) were retained in the LASSO model (FIG. 7).

The result shows that the sensitivity of the training set was 90.0%, the specificity was 100.0%, and the accuracy was 97.7% (Table 2).

The treatment method of melanoma cancer cells metastasis in lymph nodes includes, but is not limited to lymphadenectomy, chemotherapy, radiotherapy, targeting, immunotherapy, or a combination thereof.

### Example 2.3 Identifying Normal Skin Tissue and Basal Cell Carcinoma

One or more of the operations shown in Example 2.3 are the same as or similar to those explained with respect to Example 1.1, and the detailed explanation may be omitted. The differences between Example 2.3 and Example 1.1 are that specimens were obtained from normal skin tissue and basal cell carcinoma (BCC); LASSO regression was chosen for the algorithm model. Please refer to Table 2 below for detailed amount of specimen. The lymph node tissues of this example were obtained from surgery. The specimens were placed in 150 mM cold ammonium formate for 15 min, then placed on filter paper. The MiniMaP platform was also used in the example to analyze the mass spectrum, and mass spectra from surgical tissue were collected in positive ion mode in the mass range of m/z 500 to 1000. Then the training set of the example was trained through 5-fold cross validation to distinguish normal skin tissue or basal cell carcinoma from mass spectra. Finally, 29 features (m/z 846.5, m/z 719.5, m/z 751.5, m/z 774.5, m/z 780.5, m/z 814.5, m/z 813.5, m/z 759.5, m/z 766.5, m/z 692.5, m/z 888.5, m/z 731.5, m/z 677.5, m/z 704.5, m/z 772.5, m/z 804.5, m/z 744.5, m/z 781.5, m/z 702.5, m/z 716.5, m/z 830.5, m/z 908.5, m/z 783.5, m/z 696.5, m/z 890.5, m/z 896.5, m/z 784.5, m/z 912.5, and m/z 826.5) were retained in the LASSO model.

The result shows that the sensitivity of the training set was 90.5%, the specificity was 90.5%, and the accuracy was 90.5% (Table 2).

The treatment method of basal cell carcinoma includes, but is not limited to surgery, destructive methods (such as cryotherapy, radiation, laser, electrodesiccation, curettage), topical medications (pure drugs or combined with lasers of specific wavelengths, such as photodynamic therapy), or a combination thereof.

### Example 2.3.1 Performing Feature Selection and then Performing Machine Learning to Identify Normal Skin Tissue or Basal Cell Carcinoma

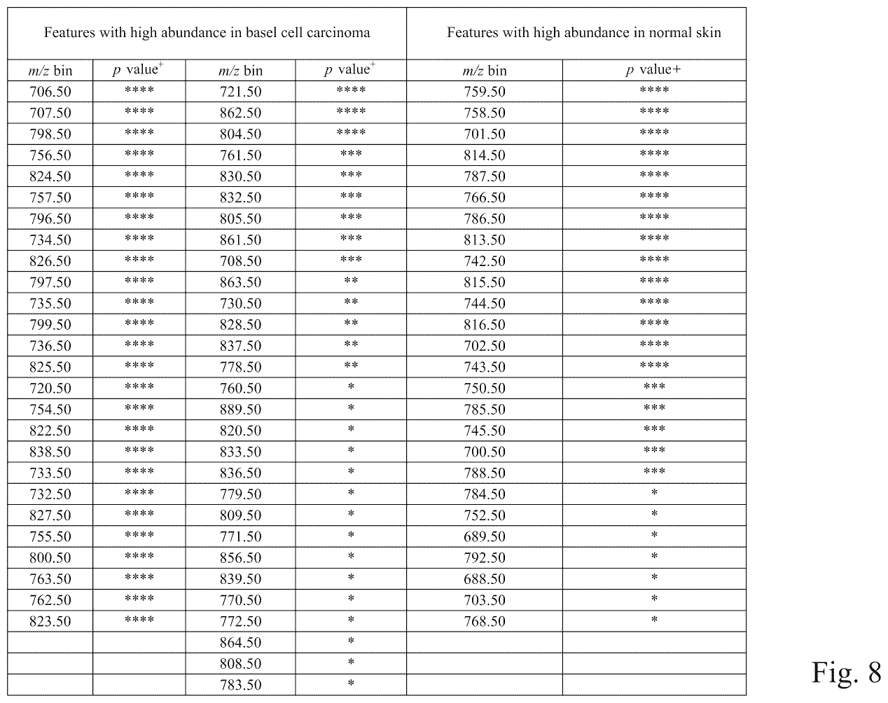

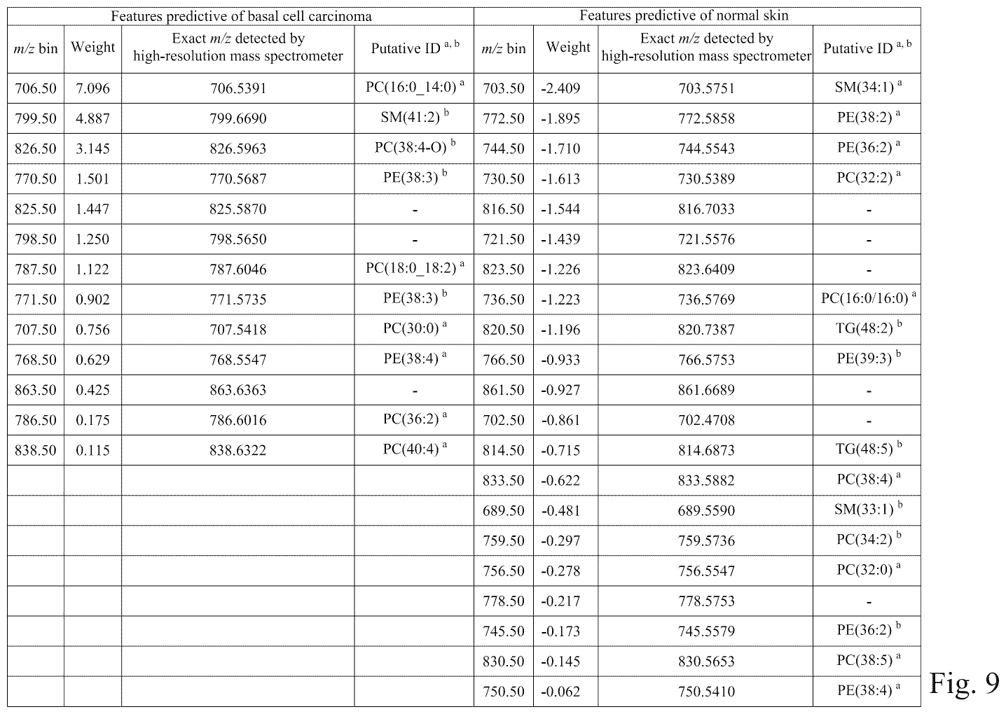

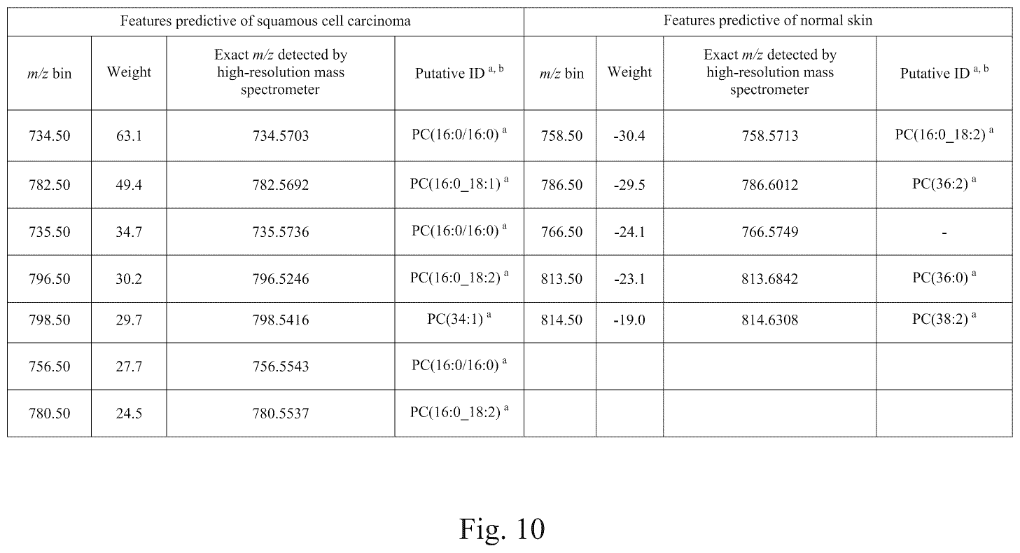

One or more of the operations shown in Example 2.3.1 are the same as or similar to those explained with respect to Example 2.3, and the detailed explanation may be omitted. The differences between Example 2.3.1 and Example 2.3 are that initial feature selection method was applied to the mass spectral data, where features were evaluated for statistical difference to obtain a plurality of initial markers, and then the plurality of initial markers were inputted into the LASSO model of the machine learning algorithm to obtain a plurality of markers. Specifically, the initial feature selection was performed by identifying features with significant differences (p<0.05) between normal skin and BCC (calculated by unpaired Student's t-test). There were initially 494 features without feature selection, and 81 features remained after the initial feature selection (FIG. 8). Next, these 81 m/z bins were used to build the model. Next, the training set of the example was trained through 10-fold cross validation to distinguish normal skin tissue or basal cell carcinoma from mass spectra. Finally, 34 features (m/z 706.5, m/z 799.5, m/z 826.5, m/z 770.5, m/z 825.5, m/z 798.5, m/z 787.5, m/z 771.5, m/z 707.5, m/z 768.5, m/z 863.5, m/z 786.5, m/z 838.5, m/z 703.5, m/z 772.5, m/z 744.5, m/z 730.5, m/z 816.5, m/z 721.5, m/z 823.5, m/z 736.5, m/z 820.5, m/z 766.5, m/z 861.5, m/z 702.5, m/z 814.5, m/z 833.5, m/z 689.5, m/z 759.5, m/z 756.5, m/z 778.5, m/z 745.5, m/z 830.5, and m/z 750.5) were retained in the LASSO model (FIG. 9).

The result shows that the sensitivity of the training set was 95.3%, the specificity was 92.9%, and the accuracy was 93.3% (Table 2).

Table 1.1 below shows a comparison of the model construction with all 494 features and 81 features that were significantly different between normal skin and BCC (FIG. 8).

TABLE 1.1

| Number of features | | | | |
|---|---|---|---|---|
| used for LASSO | | | | |
| construction | Data set | Sensitivity | Specificity | Accuracy |
| 494 features | training set | 90.5% | 90.5% | 90.5% |
| (no initial feature | | (19/21) | (76/84) | (95/105) |
| selection) | validation | 100.0% | 94.4% | 95.6% |
| | set | (9/9) | (34/36) | (51/53) |
| 81 features | training set | 95.3% | 92.9% | 93.3% |
| (initial feature | | (20/21) | (78/84) | (98/105) |
| selection performed) | validation | 100.0% | 97.2% | 97.8% |
| | set | (9/9) | (35/36) | (52/53) |

### Example 2.4 Identifying Normal Skin Tissue and Squamous Cell Carcinoma

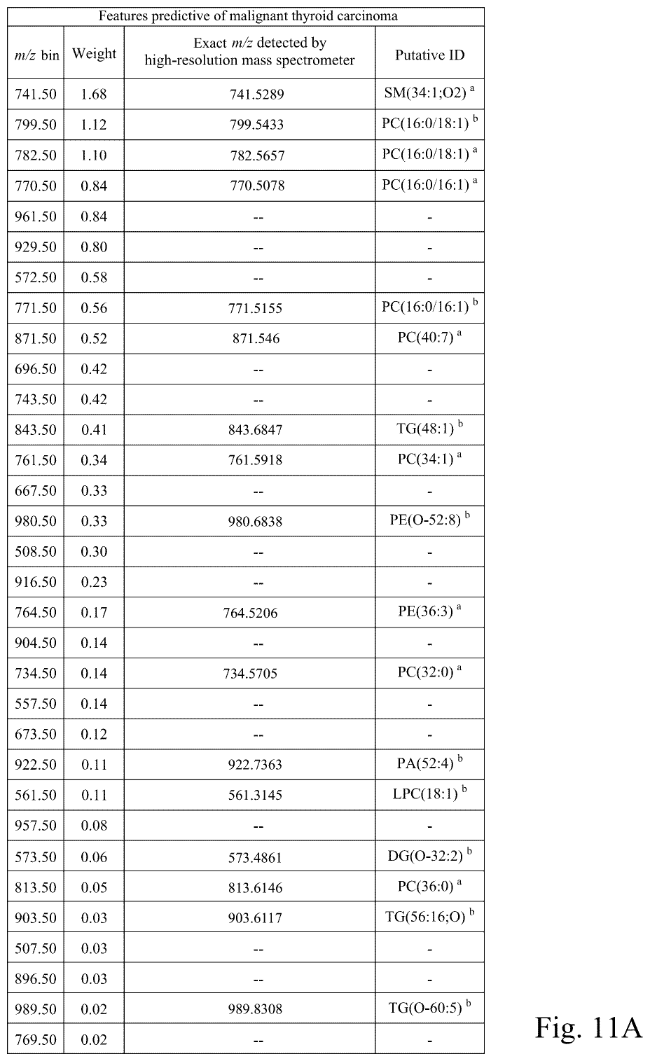

One or more of the operations shown in Example 2.4 are the same as or similar to those explained with respect to Example 1.1, and the detailed explanation may be omitted. The differences between Example 2.4 and Example 1.1 are that specimens were obtained from normal skin tissue and squamous cell carcinoma (SCC); SVM was chosen for the algorithm model. Please refer to Table 2 below for detailed amount of specimen. The squamous cell carcinoma of this example was obtained from surgery. The specimens were placed in 150 mM cold ammonium formate for 15 minutes, then placed on the filter paper. The MiniMaP platform was also used in the example to analyze the mass spectrum, and mass spectra from surgical tissue were collected in positive ion mode in the mass range of m/z 500 to 1000. Then the training set of the example was trained through 5-fold cross validation to distinguish normal skin tissue or squamous cell carcinoma from mass spectra. Finally, 12 features (m/z 734.5, m/z 782.5, m/z 735.5, m/z 796.5, m/z 798.5, m/z 756.5, m/z 780.5, m/z 758.5, m/z 786.5, m/z 766.5, m/z 813.5, and m/z 814.5) were retained in the SVM-RFE model (FIG. 10).

The result shows that the sensitivity of the training set was 100.0%, the specificity was 97.6%, and the accuracy was 97.9% (Table 2).

The treatment method of squamous cell carcinoma includes, but is not limited to surgery, destructive methods (such as cryotherapy, radiation, laser, electrodesiccation, curettage), topical medications (pure drugs or combined with lasers of specific wavelengths, such as photodynamic therapy) or a combination thereof.

### Example 3.1 Identifying Benign or Malignant Thyroid Nodule

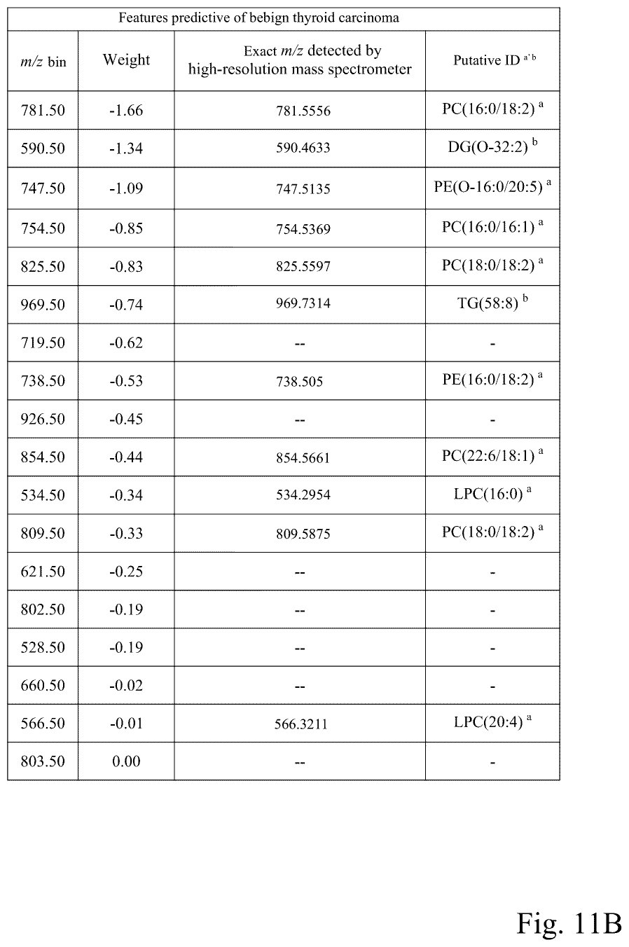

One or more of the operations shown in Example 3.1 are the same as or similar to those explained with respect to Example 1.1, and the detailed explanation may be omitted. The differences between Example 3.1 and Example 1.1 are that specimens were obtained from benign or malignant thyroid nodule; LASSO regression was chosen for the algorithm model. Please refer to Table 2 below for detailed amount of specimen. The specimens of this example were obtained from fine needle aspiration biopsy (FNB). The specimens were placed on filter paper. The MiniMaP platform was also used in the example to analyze the mass spectrum, and mass spectra from surgical tissue were collected in positive ion mode in the mass range of m/z 500 to 1000. Then the training set of the example was trained through 10-fold cross validation to distinguish benign or malignant thyroid nodule from mass spectra. Finally, 50 features (m/z 741.5, m/z 799.5, m/z 782.5, m/z 770.5, m/z 961.5, m/z 929.5, m/z 572.5, m/z 771.5, m/z 871.5, m/z 696.5, m/z 743.5, m/z 843.5, m/z 761.5, m/z 667.5, m/z 980.5, m/z 508.5, m/z 916.5, m/z 764.5, m/z 904.5, m/z 734.5, m/z 557.5, m/z 673.5, m/z 922.5, m/z 561.5, m/z 957.5, m/z 573.5, m/z 813.5, m/z 903.5, m/z 507.5, m/z 896.5, m/z 989.5, m/z 769.5, m/z 803.5, m/z 566.5, m/z 660.5, m/z 528.5, m/z 802.5, m/z 621.5, m/z 809.5, m/z 534.5, m/z 854.5, m/z 926.5, m/z 738.5, m/z 719.5, m/z 969.5, m/z 825.5, m/z 754.5, m/z 747.5, m/z 590.5, and m/z 781.5) were retained in the LASSO model (FIGS. 11A to 11B).

The result shows that the sensitivity of the training set was 94.7%, the specificity was 80.3%, and the accuracy was 89.7% (Table 2).

The treatment method of benign thyroid nodule includes, but is not limited to thyroid hormone suppression therapy, ethanol (alcohol) injection, thermal ablation, surgery, or a combination thereof. The treatment method of malignant thyroid nodule includes, but is not limited to radioactive iodine (1-131) therapy, surgery, thyroid stimulating hormone (TSH) suppression therapy, chemotherapy, or a combination thereof.

### Example 3.2 Identifying Follicular Thyroid Carcinoma (FTC) or Papillary Thyroid Carcinoma (PTC)

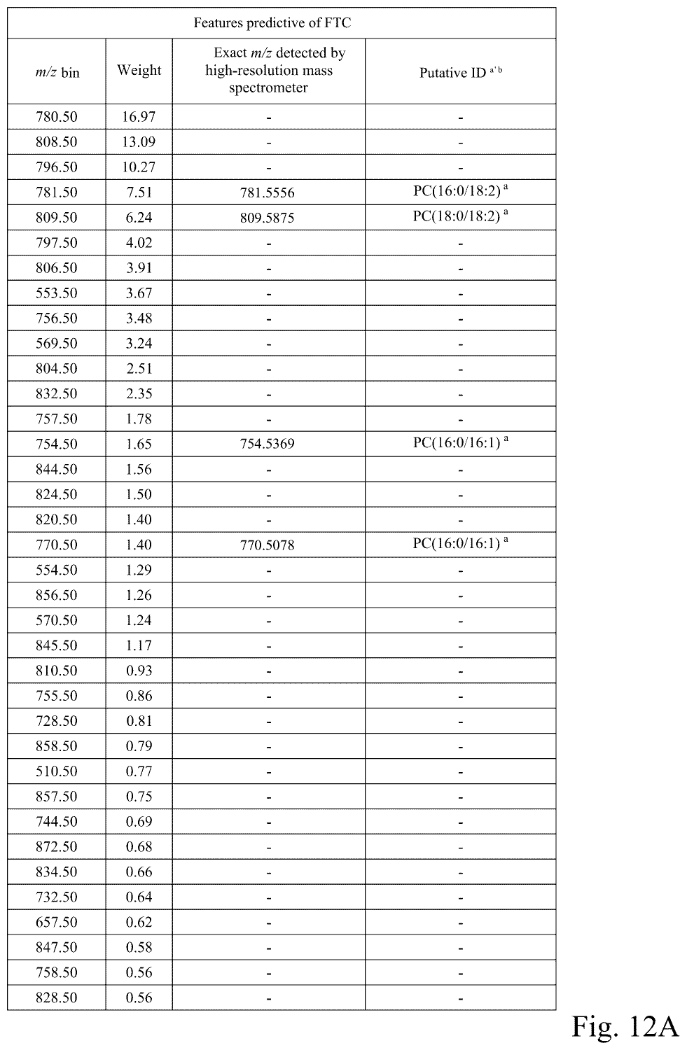

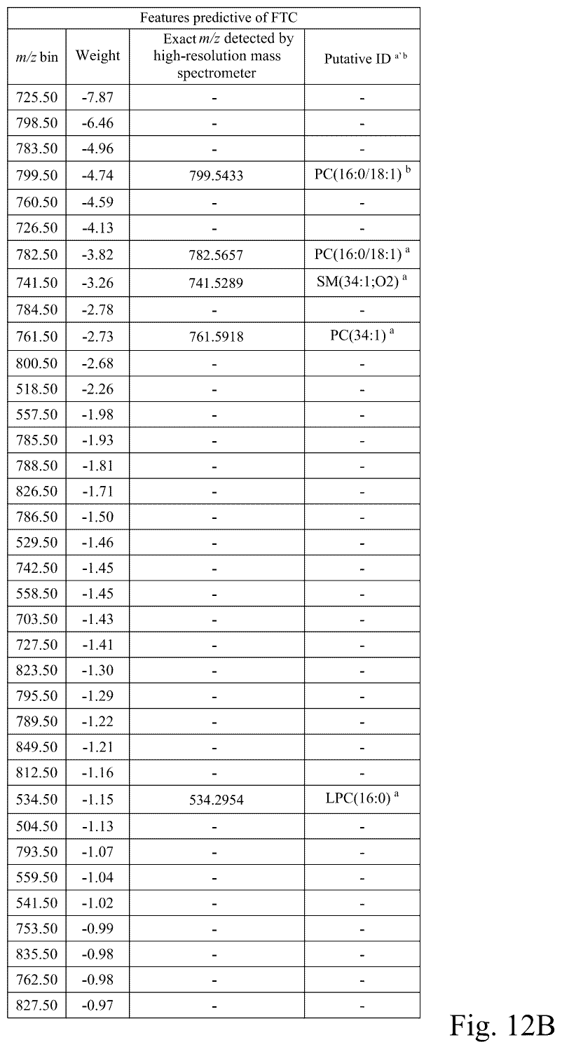

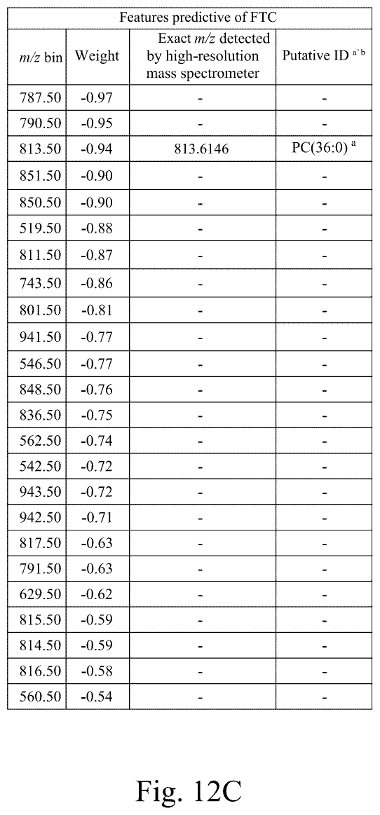

One or more of the operations shown in Example 3.2 are the same as or similar to those explained with respect to Example 1.1, and the detailed explanation may be omitted. The differences between Example 3.2 and Example 1.1 are that specimens were obtained from follicular thyroid carcinoma and papillary thyroid carcinoma tissue. Please refer to Table 2 below for detailed amount of specimen. The follicular thyroid carcinoma and papillary thyroid carcinoma tissue of this example were obtained from fine needle aspiration biopsy (FNB). The specimens were placed on filter paper. The MiniMaP platform was also used in the example to analyze the mass spectrum, and mass spectra from biopsies were collected in positive ion mode in the mass range of m/z 500 to 1000. Then the training set of the example was trained through 4-fold cross validation, and C was set to 30 for further optimization of SVM-RFE to adjust the optimal hyper-parameters to distinguish follicular thyroid carcinoma and papillary thyroid carcinoma tissue from mass spectra. Finally, 96 features were retained in the SVM-RFE model (FIGS. 12A to 12C).

The result shows that the accuracy was 91.4% (Table 2).

The treatment method of follicular thyroid carcinoma includes, but is not limited to surgery, radiotherapy, chemotherapy, hormone therapy, targeted therapy, immunotherapy, or a combination thereof. The treatment method of papillary thyroid carcinoma includes, but is not limited to surgery, radiotherapy, chemotherapy, hormone therapy, targeted therapy, immunotherapy, or a combination thereof.

TABLE 2

| | | Samples | | | | |
|---|---|---|---|---|---|---|
| | | collected | | Training set performance | | |
| | Purpose | and analyzed | | Sensitivity | Specificity | Accuracy |
| Breast | Rapid diagnosis | Benign | 665 | 80.5% | 90.3% | 87.5% |
| cancer | of core needle | CNB | | | | |
| | biopsies (CNB) | Malignant | 333 | | | |
| | Subtype | CNB | | — | — | 75.3% |
| | determination | | | | | |
| | in CNBs | | | | | |
| | Prediction of | Normal | 244 | 77.8% | 90.8% | 88.8% |
| | metastasis | LNs | | | | |
| | in lymph | Metastatic | 113 | | | |
| | nodes (LNs) | LNs | | | | |
| | Surgical | Normal | 198 | 86.5% | 95.3% | 91.2% |
| | margin | Tissue | | | | |
| | evaluation | Tumor | 206 | | | |
| | with tissue- | | | | | |
| | stained filter | | | | | |
| | paper | | | | | |
| Skin | Rapid | Normal | 128 | 87.5% | 92.9% | 91.7% |
| Cancer | melanoma | skin | | | | |
| | diagnosis | Melanoma | 55 | | | |
| | | Benign | 8 | | | |
| | | Nevus | | | | |
| | Prediction of | Normal | 34 | 90.0% | 100.0% | 97.7% |
| | metastasis | LNs | | | | |
| | in lymph | Metastatic | 10 | | | |
| | nodes (LNs) | LNs | | | | |
| | Basal cell | Normal | 120 | 95.3% | 92.9% | 93.3% |
| | carcinoma | skin | | | | |
| | (BCC) | BCC | 30 | | | |
| | diagnosis | | | | | |
| | Squamous | Normal | 120 | 100.0% | 97.6% | 97.9% |
| | cell | skin | | | | |
| | carcinoma | SCC | 15 | | | |
| | (SCC) | | | | | |
| | diagnosis | | | | | |
| Thyroid | Determining | Benign | 112 | 94.7% | 80.3% | 89.7% |
| Cancer | malignancy in | FNB | | | | |
| | indeterminate | Malignant | 203 | | | |
| | fine needle | FNB | | | | |
| | biopsies | | | | | |
| | (FNBs) | | | | | |
| | Distinguishing | PTC | 196 | — | — | 91.4% |
| | follicular | FTC | 8 | | | |
| | thyroid | | | | | |
| | carcinoma | | | | | |
| | (FTC) from | | | | | |
| | papillary | | | | | |
| | thyroid | | | | | |
| | carcinoma | | | | | |
| | (PTC) | | | | | |

### Example 4.1 Using Breast Cancer Biomarkers from Previously Constructed Machine Learning Model to Distinguish Benign Breast Tumor and Malignant Breast Tumor

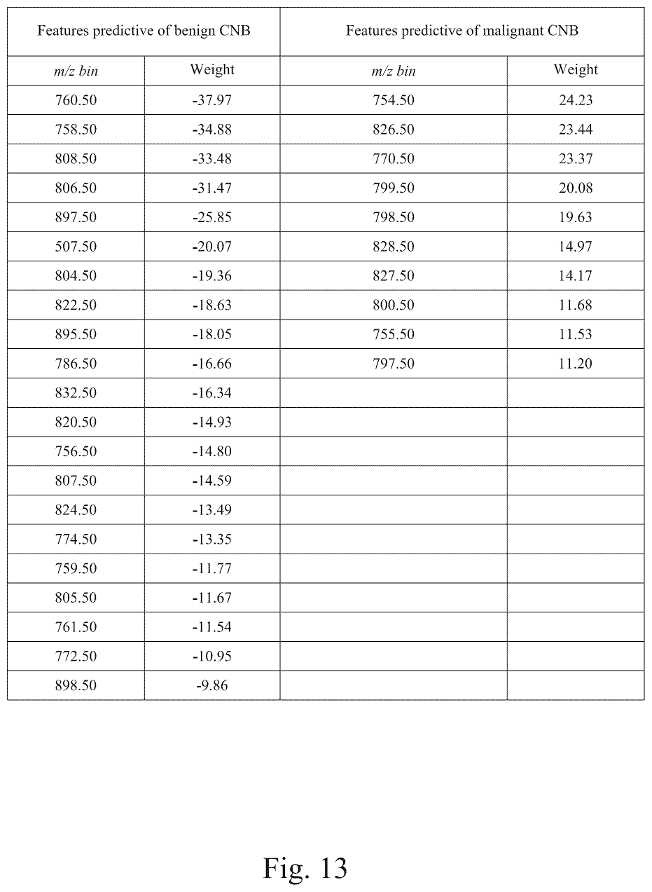

A SVM-RFE machine learning model was previously constructed using data acquired from Waters™ Synapt® G2 (a high-resolution mass spectrometer). A total of 29 benign CNB and 26 malignant CNB (from 180 samples of training set and testing set in Table 1) was normalized to the TIC, and the average mass spectral data with a bin of 1 Da was acquired. Data was split into training and testing data at a 7:3 ratio for machine learning model construction. The model achieved a 84.2% accuracy, 77.8% sensitivity and 90.0% specificity in 10-fold cross validation of the training set (Table 3). 31 features were selected (FIG. 13) as a known feature and used to predict the validation set in Example 1.1.

Prediction of the 512 CNBs (from 512 samples of validation set in Table 1) in the validation set revealed 80.9% accuracy, 71.2% sensitivity, and 85.0% specificity. These results are comparable with the performance of the previously constructed machine learning model, suggesting the applicability of known feature sets or established databases for the prediction of data acquired using MiniMaP.

TABLE 3

| | Data set | Sensitivity | Specificity | Accuracy |
|---|---|---|---|---|
| Data from high- | Training | 77.8% | 90.0% | 84.2% |
| resolution mass | set | (14/18) | (18/20) | (32/38) |
| spectrometer | Testing | 75.0% | 88.9% | 82.4% |
| (m/z bin size of 1 Da) | set | (6/8) | (8/9) | (14/17) |
| Data from low- | Validation | 71.2% | 85.0% | 80.9% |
| resolution mass | set | (109/153) | (305/359) | (414/512) |
| spectrometer | | | | |
| (m/z bin size of 1 Da) | | | | |

### Example 4.2 Using Literature Reported Features for Classification of Breast Tumor and Malignant Breast Tumor

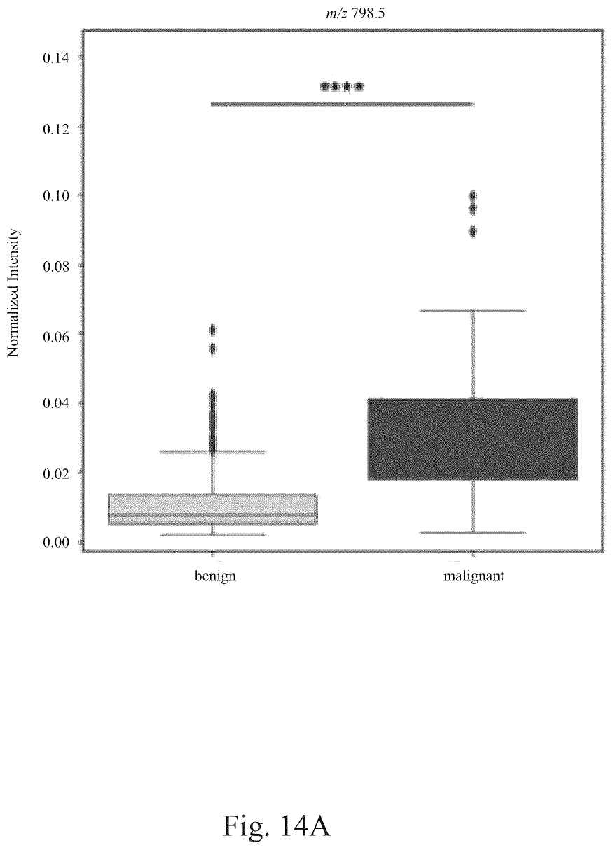

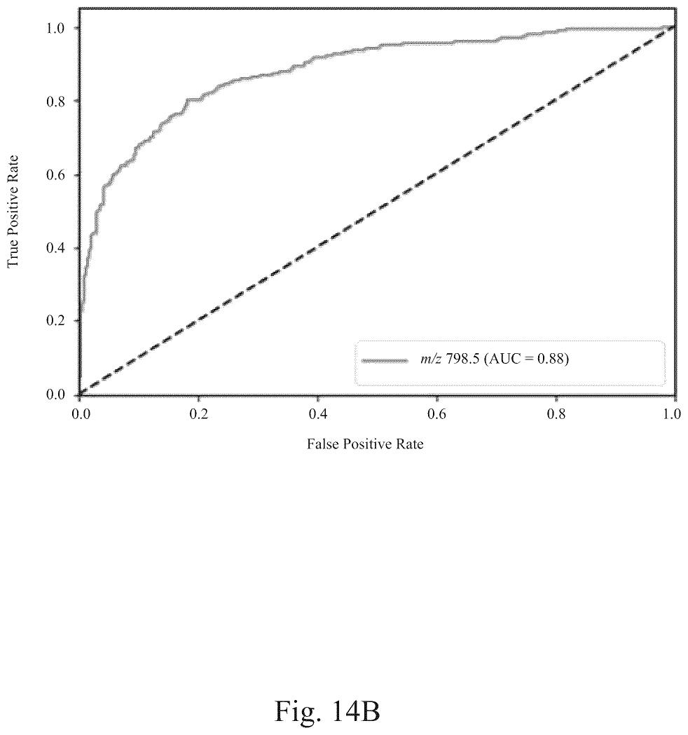

Research is ongoing to discover biomarkers related to cancers. For example, the abundance of compound PC (34:1) was reported to differ in malignant and benign breast tumors. This compound, M, could potentially have adducts of hydrogen [M+H]⁺, sodium [M+Na]⁺, and potassium [M+K]⁺, upon ionization. Based on its exact mass of 759.5778, the compounds would be respectively in m/z bin 760.5, m/z bin 782.5 and m/z bin 798.5. For the purpose of demonstrating the feasibility of using literature reported features for classification, only the adduct m/z bin 798.5 which has higher abundance will be discussed. Using the benign and malignant samples in Table 1 (total of 692 samples), the boxplot (FIG. 14A) shows significant difference in the abundance of m/z bin 798.5.

Average AUC of the ROC curve was 0.88 when using m/z bin 798.5 to distinguish benign and malignant tumor (FIG. 14B). These results suggest the potential of using literature reported features, for example a single m/z bin, for the prediction of data acquired using MiniMaP.

The present disclosure provides an economical, easy-to-operate method of establishing cancer screening module, using method and platform thereof for medical personnel and non-professionals. This technology uses a low-resolution mass spectrometer combined with a simple-to-operate screening platform designed by machine learning algorithm to quickly screen for cancer. Intraoperative and treatment decisions can be made more quickly through non-targeted metabolomics methods. At the same time, it can also reduce the psychological burden of patients while waiting for reports, and is widely used in medical screening.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of establishing cancer screening module, comprising:
providing a plurality of samples and a plurality of cancer statuses corresponding to the plurality of samples;
analyzing the plurality of samples with a low-resolution mass spectrometer to obtain a plurality of mass spectral data, wherein the low-resolution mass spectrometer is a mass spectrometer mass accuracy level above 5 ppm and a mass resolution below 10,000 m/Δm;
inputting the plurality of mass spectral data into a machine learning algorithm to obtain a plurality of markers by a feature selection method; and
using the plurality of markers and the plurality of cancer statuses to establish the cancer screening model by the machine learning algorithm.

2. The method of claim 1, wherein the inputting the plurality of mass spectral data into the machine learning algorithm comprises:
obtaining a plurality of initial markers from the plurality of mass spectral data by an initial feature selection method, and then inputting the plurality of initial markers into the machine learning algorithm, wherein the initial feature selection method comprises filter method.

3. The method of claim 1, wherein when the feature selection method is wrapper method or embedded method, the feature selection method comprises splitting the plurality of samples into a training set and a validation set, calculating a sensitivity, a specificity, an accuracy, an area under curve (AUC) of a receiver operating characteristic (ROC), or a combination thereof to obtain the plurality of markers.

4. The method of claim 3, wherein the wrapper method comprises a recursive feature elimination (RFE) to obtain the plurality of markers.

5. The method of claim 1, before the inputting the plurality of mass spectral data into the machine learning algorithm, the method further comprising performing a normalized preprocessing on the plurality of mass spectral data, the normalization preprocessing comprising: normalization, m/z alignment, average MS spectra, m/z binning, noise removal, data scaling, or a combination thereof.

6. The method of claim 5, wherein the m/z binning comprises binning by size from 0.5 daltons to 1.5 daltons.

7. The method of claim 1, wherein the low-resolution mass spectrometer is single quadrupole mass spectrometer, wherein the mass accuracy level is from 5 ppm to 1200 ppm and the mass resolution is below 10,000 m/Δm.

8. The method of claim 1, wherein the machine learning algorithm comprises kernel-based, regression, tree-based, dimension reduction, probabilistic, distance-based, or any combination thereof.

9. The method of claim 8, wherein the kernel-based comprises support vector machine (SVM),
when the plurality of samples is a plurality of benign breast tumor samples and a plurality of malignant breast tumor samples, the SVM is used to analyze the plurality of cancer statuses being benign breast tumor or malignant breast tumor;
when the plurality of samples are a plurality of HR negative breast cancer tumor samples and a plurality of HR positive breast cancer tumor samples, the SVM is used to analyze the plurality of cancer statuses being HR negative breast cancer tumor or HR positive breast cancer tumor;
when the plurality of samples are a plurality of HER2 negative breast cancer tumor samples and a plurality of HER2 positive breast cancer tumor samples, the SVM is used to analyze the plurality of cancer statuses being HER2 negative breast cancer tumor or HER2 positive breast cancer tumor;
when the plurality of samples comprise a plurality of HR negative breast cancer tumor samples, a plurality of HER2 negative breast cancer tumor samples, a plurality of HR positive breast cancer tumor samples and a plurality of HER2 positive breast cancer tumor samples, the SVM is used to analyze the plurality of cancer statuses being HR negative HER2 negative breast cancer tumor, HR negative HER2 positive breast cancer tumor, HR positive HER2 negative breast cancer tumor, or HR positive HER2 positive breast cancer tumor;
when the plurality of samples are a plurality of normal lymph node samples and a plurality of samples of lymph node with metastatic breast cancer cells, the SVM is used to analyze the plurality of cancer statuses being lymph node without breast cancer metastasis or lymph node with breast cancer metastasis;
when the plurality of samples are a plurality of samples of normal surgical margins of the breast and a plurality of breast cancer tissue samples, the SVM is used to analyze the plurality of cancer statuses being normal breast tissue or breast cancer tissue;
when the plurality of samples are a plurality of normal skin tissue samples and squamous cell carcinoma samples, the SVM is used to analyze the plurality of cancer statuses being normal skin tissue or squamous cell carcinoma; or
when the plurality of samples are a plurality of follicular thyroid carcinoma samples and a plurality of papillary thyroid carcinoma samples, the SVM is used to analyze the plurality of cancer statuses being follicular thyroid carcinoma or papillary thyroid carcinoma.

10. The method of claim 9,
wherein when the plurality of samples are the plurality of benign breast tumor samples and the plurality of malignant breast tumor samples, the SVM comprises SVM-RFE to obtain the plurality of markers,
wherein the plurality of markers are selected from any one or any combination of the group consisting of m/z 782.5, m/z 798.5, m/z 754.5, m/z 770.5, m/z 923.5, m/z 772.5, m/z 757.5, m/z 774.5, m/z 788.5, m/z 753.5, and m/z 727.5.

11. The method of claim 9,
wherein when the plurality of samples are the plurality of samples of normal surgical margins of the breast and the plurality of breast cancer tumor samples, the SVM comprises SVM-RFE to obtain the plurality of markers,
wherein the plurality of markers are selected from any one or any combination of the group consisting of m/z 504.5, m/z 835.5, m/z 575.5, m/z 723.5, m/z 764.5, m/z 837.5, m/z 804.5, m/z 547.5, m/z 853.5, m/z 765.5, m/z 788.5, m/z 828.5, m/z 781.5, m/z 727.5, m/z 759.5, m/z 836.5, m/z 682.5, m/z 753.5, m/z 786.5, m/z 770.5, m/z 805.5, m/z 518.5, m/z 768.5, m/z 755.5, m/z 782.5, m/z 756.5, m/z 824.5, m/z 754.5, m/z 647.5, and m/z 848.5.

12. The method of claim 9,
wherein when the plurality of samples are the plurality of normal lymph node samples and the plurality of samples of lymph nodes with metastatic breast cancer cells, the SVM comprises SVM-RFE to obtain the plurality of markers,
wherein the plurality of markers are selected from any one or any combination of the group consisting of m/z 509.5, m/z 531.5, m/z 534.5, m/z 567.5, m/z 575.5, m/z 615.5, m/z 641.5, m/z 643.5, m/z 698.5, m/z 742.5, m/z 754.5, m/z 758.5, m/z 761.5, m/z 781.5, m/z 782.5, m/z 797.5, m/z 798.5, m/z 805.5, m/z 820.5, m/z 824.5, m/z 828.5, m/z 829.5, m/z 830.5, m/z 846.5, m/z 879.5, and m/z 880.5.

13. The method of claim 9,
wherein when the plurality of samples are the plurality of normal skin tissue samples and the plurality of squamous cell carcinoma samples, the SVM comprises SVM-RFE to obtain the plurality of markers,
wherein the plurality of markers are selected from any one or any combination of the group consisting of m/z 734.5, m/z 782.5, m/z 735.5, m/z 796.5, m/z 798.5, m/z 756.5, m/z 780.5, m/z 758.5, m/z 786.5, m/z 766.5, m/z 813.5, and m/z 814.5.

14. The method of claim 8, wherein the regression comprises LASSO regression,
when the plurality of samples are a plurality of normal skin tissue samples, a plurality of benign nevus tissue samples and a plurality of melanoma samples, the LASSO regression is used to analyze the plurality of cancer statuses being normal skin tissue or melanoma;
when the plurality of samples are a plurality of normal lymph node samples and a plurality of samples of lymph nodes with metastatic melanoma cells, the LASSO regression is used to analyze the plurality of cancer statuses being lymph node without metastatic melanoma or lymph node with metastatic melanoma;
when the plurality of samples are a plurality of normal skin tissue samples and a plurality of basal cell carcinoma samples, the LASSO regression is used to analyze the plurality of cancer statuses being normal skin tissue or basal cell carcinoma; or
when the plurality of samples are a plurality of benign thyroid nodule samples and a plurality of malignant thyroid nodule samples, the LASSO regression is used to analyze the plurality of cancer statuses being benign thyroid nodule or malignant thyroid nodule.

15. The method of claim 14, wherein when the plurality of samples are the plurality of normal skin tissue samples, the plurality of benign nevus tissue samples and the plurality of melanoma samples, the LASSO regression comprises LASSO regression feature selection to obtain the plurality of markers,
wherein the plurality of markers are selected from any one or any combination of the group consisting of m/z 766.5, m/z 664.5, m/z 728.5, m/z 729.5, m/z 773.5, m/z 719.5, m/z 692.5, m/z 752.5, m/z 757.5, m/z 736.5, m/z 862.5, m/z 672.5, m/z 603.5, m/z 832.5, and m/z 521.5.

16. The method of claim 14, wherein when the plurality of samples are the plurality of normal lymph node samples and the plurality of samples of lymph node with metastatic melanoma cells, the LASSO regression comprises LASSO regression feature selection to obtain the plurality of markers,
wherein the plurality of markers are selected from any one or any combination of the group consisting of m/z 700.5, m/z 761.5, m/z 771.5, m/z 732.5, m/z 708.5, m/z 817.5, and m/z 622.5.

17. The method of claim 14,
wherein when the plurality of samples are the plurality of normal skin tissue samples and the plurality of basal cell carcinoma samples, the LASSO regression comprises LASSO regression feature selection to obtain the plurality of markers,
wherein the plurality of markers are selected from any one or any combination of the group consisting of m/z 846.5, m/z 719.5, m/z 751.5, m/z 774.5, m/z 780.5, m/z 814.5, m/z 813.5, m/z 759.5, m/z 766.5, m/z 692.5, m/z 888.5, m/z 731.5, m/z 677.5, m/z 704.5, m/z 772.5, m/z 804.5, m/z 744.5, m/z 781.5, m/z 702.5, m/z 716.5, m/z 830.5, m/z 908.5, m/z 783.5, m/z 696.5, m/z 890.5, m/z 896.5, m/z 784.5, m/z 912.5, and m/z 826.5.

18. The method of claim 14, wherein the inputting the plurality of mass spectral data into the machine learning algorithm comprises:
obtaining a plurality of initial markers from the plurality of mass spectral data by an initial feature selection method, and then inputting the plurality of initial markers into the machine learning algorithm, wherein the initial feature selection method comprises filter method,
wherein when the plurality of samples are the plurality of normal skin tissue samples and the plurality of basal cell carcinoma samples, the LASSO regression comprises LASSO regression feature selection to obtain the plurality of markers,
wherein the plurality of markers are selected from any one or any combination of the group consisting of m/z 706.5, m/z 799.5, m/z 826.5, m/z 770.5, m/z 825.5, m/z 798.5, m/z 787.5, m/z 771.5, m/z 707.5, m/z 768.5, m/z 863.5, m/z 786.5, m/z 838.5, m/z 703.5, m/z 772.5, m/z 744.5, m/z 730.5, m/z 816.5, m/z 721.5, m/z 823.5, m/z 736.5, m/z 820.5, m/z 766.5, m/z 861.5, m/z 702.5, m/z 814.5, m/z 833.5, m/z 689.5, m/z 759.5, m/z 756.5, m/z 778.5, m/z 745.5, m/z 830.5, and m/z 750.5.

19. The method of claim 14, wherein when the plurality of samples are the plurality of benign thyroid nodule samples and the plurality of malignant thyroid nodule samples, the LASSO regression comprises LASSO regression feature selection to obtain the plurality of markers,
wherein the plurality of markers are selected from any one or any combination of the group consisting of m/z 741.5, m/z 799.5, m/z 782.5, m/z 770.5, m/z 961.5, m/z 929.5, m/z 572.5, m/z 771.5, m/z 871.5, m/z 696.5, m/z 743.5, m/z 843.5, m/z 761.5, m/z 667.5, m/z 980.5, m/z 508.5, m/z 916.5, m/z 764.5, m/z 904.5, m/z 734.5, m/z 557.5, m/z 673.5, m/z 922.5, m/z 561.5, m/z 957.5, m/z 573.5, m/z 813.5, m/z 903.5, m/z 507.5, m/z 896.5, m/z 989.5, m/z 769.5, m/z 803.5, m/z 566.5, m/z 660.5, m/z 528.5, m/z 802.5, m/z 621.5, m/z 809.5, m/z 534.5, m/z 854.5, m/z 926.5, m/z 738.5, m/z 719.5, m/z 969.5, m/z 825.5, m/z 754.5, m/z 747.5, m/z 590.5, and m/z 781.5.

20. The method of claim 1, wherein the analyzing the plurality of samples with the low-resolution mass spectrometer comprises:
ionizing the plurality of samples by a paper spray ionization (PSI) method; and
analyzing the plurality of samples from the plurality of ionized samples by the low-resolution mass spectrometer.

21. The method of claim 20, wherein the PSI method comprises:
using a PSI device comprising:
a base;
a solvent rack disposed above the base; and
a clamp having a fixing end and a clamping end, the fixing end disposed on the base;
placing one of a plurality of paper sheets to a clamping end of the clamp;
placing a solvent to the solvent rack of the PSI device;
placing the plurality of samples on the different plurality of paper sheets, and using the solvent to perform PSI to obtain a plurality of ionized substance; and
collecting the plurality of ionized substance and analyzing the plurality of samples with the low-resolution mass spectrometer.

22. The method of claim 20, wherein the PSI method comprises:
using a PSI device, the PSI device comprising:
a base;
an abutting member disposed on the base;
a loading plate movably disposed on the base, the loading plate comprising:
a body having a bottom surface and a side surface adjacent to the bottom surface, the side surface movably abutting the abutting member, and the bottom surface movably abutting the base;
a protrusion protruding outward from the bottom surface of the body; and
a metal placing piece disposed on the body and the protrusion;
a solvent rack disposed on the base; and
placing one of a plurality of paper sheets on the metal placing piece, and a corner of the one of the plurality of paper sheets protruding outward from the protrusion, and a protruding direction of the corner and a protruding direction of the protrusion being the same and facing toward the base;
placing a solvent to the solvent rack of the PSI device;
placing the plurality of samples on the different plurality of paper sheets respectively, and using the solvent to perform PSI to obtain a plurality of ionized substance; and
collecting the plurality of ionized substance and analyzing the plurality of samples with the low-resolution mass spectrometer.

23. A method for cancer screening using the cancer screening model of claim 1, comprising:
providing a specimen of a subject;
analyzing the specimen by the low-resolution mass spectrometer to obtain a subject mass spectral data; and
inputting the subject mass spectral data into the cancer screening model to perform calculation, comparison, and evaluating a risk of a cancer for the subject.

24. The method of claim 23, wherein the cancer comprises breast cancer, thyroid cancer, or skin cancer.

25. A cancer screening platform, comprising:
a low-resolution mass spectrometer configured to analyze a plurality of samples to obtain a plurality of mass spectral data, wherein the low-resolution mass spectrometer is a mass spectrometer with mass accuracy level above 5 ppm and a mass resolution below 10,000 m/Δm; and
a cancer screening model comprising a computer processor and a memory, the memory storing a plurality of computer program instructions that, when executed by the computer processor, cause the computer processor to implement following steps, comprising:
inputting the plurality of mass spectral data into a machine learning algorithm to obtain a plurality of markers by a feature selection method; and
using the plurality of markers and a plurality of cancer statuses corresponding to the plurality of samples to establish the cancer screening model by the machine learning algorithm.

26. The cancer screening platform of claim 25, further comprising a PSI device, the PSI device comprising:
a base;
a paper sheet;
a clamp having a fixing end and a clamping end, the fixing end disposed on the base, the clamping end clamping the paper sheet;
a solvent rack disposed on the base, and the paper sheet adjacent to the solvent rack; and
a mass spectrometry inlet located below the paper sheet.

27. The cancer screening platform of claim 25, further comprising a PSI device, the PSI device comprising:
a base;
an abutting member disposed on the base;
a loading plate movably disposed on the base, the loading plate comprising:
a body having a bottom surface and a side surface adjacent to the bottom surface, the side surface movably abutting the abutting member, and the bottom surface movably abutting the base;
a protrusion protruding outward from the bottom surface of the body; and
a metal placing piece disposed on the body and the protrusion; and
one of a plurality of paper sheets placed on the metal placing piece, and a corner of the one of the plurality of paper sheets protruding outward from the protrusion, and a protruding direction of the corner and a protruding direction of the protrusion being the same and facing toward the base;
a solvent rack disposed on the base; and
a mass spectrometry inlet located below the one of the plurality of paper sheets;
placing a solvent to the solvent rack of the PSI device;
placing the plurality of samples on the different plurality of paper sheets respectively, and using the solvent to perform PSI to obtain a plurality of ionized substance; and
collecting the plurality of ionized substance and analyzing the plurality of samples with the low-resolution mass spectrometer.

28. The cancer screening platform of claim 25, wherein the inputting the plurality of mass spectral data into the machine learning algorithm comprises:
obtaining a plurality of initial markers from the plurality of mass spectral data by an initial feature selection method, and then inputting the plurality of initial markers into the machine learning algorithm, wherein the initial feature selection method comprises filter method.

29. The cancer screening platform of claim 25, wherein when the feature selection method is wrapper method or embedded method, the feature selection method comprises splitting the plurality of samples into a training set and a validation set, calculating a sensitivity, a specificity, an accuracy, an AUC of a ROC to obtain the plurality of markers.

30. The cancer screening platform of claim 29, wherein the wrapper method comprises a RFE to obtain the plurality of markers.

31. The cancer screening platform of claim 25, before the inputting the plurality of mass spectral data into the machine learning algorithm, the method further comprising performing an normalized preprocessing on the plurality of mass spectral data, the normalization preprocessing comprising: normalization, m/z alignment, average MS spectra, m/z binning, noise removal, data scaling, or a combination thereof.

32. The cancer screening platform of claim 31, wherein the m/z binning comprises binning by size from 0.5 daltons to 1.5 daltons.

33. The cancer screening platform of claim 25, the low-resolution mass spectrometer is single quadrupole mass spectrometer, wherein the mass accuracy level is from 5 ppm to 1200 ppm and the mass resolution is below 10,000 m/Δm.

34. The cancer screening platform of claim 25, wherein the machine learning algorithm comprises kernel-based, regression, tree-based, dimension reduction, probabilistic, distance-based, or any combination thereof.

35. The cancer screening platform of claim 34, wherein the kernel-based comprises SVM,
when the plurality of samples are a plurality of benign breast tumor samples and a plurality of malignant breast tumor samples, the SVM is used to analyze the plurality of cancer statuses being benign breast tumor or malignant breast tumor;
when the plurality of samples are a plurality of normal lymph node samples and a plurality of samples of lymph node with metastatic breast cancer cells, the SVM is used to analyze the plurality of cancer statuses being lymph node without breast cancer metastasis or lymph node with breast cancer metastasis;
when the plurality of samples are a plurality of HR negative breast cancer tumor samples and a plurality of HR positive breast cancer tumor samples, the SVM is used to analyze the plurality of cancer statuses being HR negative breast cancer tumor or HR positive breast cancer tumor;
when the plurality of samples are a plurality of HER2 negative breast cancer tumor samples and a plurality of HER2 positive breast cancer tumor samples, the SVM is used to analyze the plurality of cancer statuses being HER2 negative breast cancer tumor or HER2 positive breast cancer tumor;
when the plurality of samples comprises a plurality of HR negative breast cancer tumor samples, a plurality of HER2 negative breast cancer tumor samples, a plurality of HR positive breast cancer tumor samples and a plurality of HER2 positive breast cancer tumor samples, the SVM is used to analyze the plurality of cancer statuses being HR negative HER2 negative breast cancer tumor, HR negative HER2 positive breast cancer tumor, HR positive HER2 negative breast cancer tumor, or HR positive HER2 positive breast cancer tumor;
when the plurality of samples are a plurality of samples of normal surgical margins of the breast and a plurality of breast cancer tissue samples, the SVM is used to analyze the plurality of cancer statuses being normal breast tissue or breast cancer tissue;
when the plurality of samples are a plurality of normal skin tissue samples and squamous cell carcinoma samples, the SVM is used to analyze the plurality of cancer statuses being normal skin tissue or squamous cell carcinoma; or
when the plurality of samples are a plurality of follicular thyroid carcinoma samples and a plurality of papillary thyroid carcinoma samples, the SVM is used to analyze the plurality of cancer statuses being follicular thyroid carcinoma or papillary thyroid carcinoma.

36. The cancer screening platform of claim 34, wherein the regression comprises LASSO regression,
when the plurality of samples are a plurality of normal skin tissue samples, benign nevus tissue samples and a plurality of melanoma samples, the LASSO regression is used to analyze the plurality of cancer statuses being normal skin tissue or melanoma;
when the plurality of samples are a plurality of normal lymph node samples and a plurality of metastatic melanoma cells in lymph node samples, the LASSO regression is used to analyze the plurality of cancer statuses being lymph node without metastatic melanoma or lymph node with metastatic melanoma;
when the plurality of samples are a plurality of normal skin tissue samples and basal cell carcinoma samples, the LASSO regression is used to analyze the plurality of cancer statuses being normal skin tissue or basal cell carcinoma; or
when the plurality of samples are a plurality of benign thyroid nodule samples and a plurality of malignant thyroid nodule samples, the LASSO regression is used to analyze the plurality of cancer statuses being benign thyroid nodule or malignant thyroid nodule.

37. The cancer screening platform of claim 25, wherein
the low-resolution mass spectrometer is configured to analyze a specimen to obtain a subject mass spectral data;
the cancer screening model is executed by the computer processor, cause the computer processor to implement following steps, further comprising: inputting the subject mass spectral data into the cancer screening model to perform calculation, comparison, and evaluating a risk of a cancer for the subject.

38. A cancer screening platform, comprising:
a low-resolution mass spectrometer configured to analyze a specimen of a subject to obtain a subject mass spectral data, wherein the low-resolution mass spectrometer is a mass spectrometer with mass accuracy level above 5 ppm and a mass resolution below 10,000 m/Δm; and
a cancer screening model comprising a computer processor and a memory, the memory storing a plurality of computer program instructions that, when executed by the computer processor, cause the computer processor to implement following steps, comprising:
inputting at least one known marker;
inputting the subject mass spectral data; and
comparing the at least one known marker and the subject mass spectral data to evaluating a risk of a cancer for the subject.

39. The cancer screening platform of claim 38, wherein the at least one known marker is obtain from
a high-resolution mass spectrometer being able to provide mass accuracy level below or equal to 5 ppm and a mass resolution above or equal to 10,000 (m/Δm, full width at half-maximum height, FWHM), or
a low-resolution mass spectrometer being able to provide mass accuracy level above 5 ppm and a mass resolution below to 10,000 (m/Δm, FWHM).

40. The cancer screening platform of claim 38, wherein the cancer screening model is executed by the computer processor, cause the computer processor to implement following steps, further comprising:
performing a normalized preprocessing on the subject mass spectral data, the normalization preprocessing comprising: normalization, m/z alignment, average MS spectra, m/z binning, noise removal, data scaling, or a combination thereof.

41. The cancer screening platform of claim 40, wherein the m/z binning comprises binning by size from 0.5 daltons to 1.5 daltons.
